(19) United States
(12) Patent Application Publication
(10) Pub. No.: US 2007/0196848 A1
(43) Pub. Date: Aug. 23, 2007

(54) DETECTION OF PHOSPHORYLATED eIF2-alpha AS A DIAGNOSTIC TEST FOR EFFICACY AND SENSITIVITY OF TRANSLATION INITIATION INHIBITORS IN THE TREATMENT OF CANCER AND OTHER PROLIFERATIVE DISEASES

(75) Inventors: Jose A. Halperin, Brookline, MA (US); Huseyin Aktas, Newton, MA (US)

Correspondence Address: BANNER & WITCOFF, LTD., 28 STATE STREET, 28th FLOOR, BOSTON, MA, 02109-9601, US

(73) Assignee: President and Fellows of Harvard College, Cambridge, MA (US)

(21) Appl. No.: 11/670,731

(22) Filed: Feb. 2, 2007

Related U.S. Application Data
(63) Continuation of application No. PCT/US05/28177, filed on Aug. 10, 2005.
(60) Provisional application No. 60/600,396, filed on Aug. 10, 2004.

(51) Int. Cl. C12Q 1/68; G01N 33/574

(52) U.S. Cl. 435/6; 435/7.23

(57) ABSTRACT

The present invention relates to methods for determining the effectiveness of one or more agents for treating one or more disorders associated with aberrant cellular proliferation. Screening assays for the discovery of agents that alter eIF2α phosphorylation, inhibit translation initiation and/or inhibit aberrant cellular proliferation are also provided.

### RELATED APPLICATIONS

This application is a continuation of PCT application no. PCT/US2005/028177, designating the United States and filed Aug. 10, 2005; which claims the benefit of the filing date of U.S. provisional application No. 60/600,396, filed Aug. 10, 2004; both of which are hereby incorporated herein by reference in their entirety for all purposes.

### STATEMENT OF GOVERNMENT INTERESTS

This invention was made with government support under grant number R01 CA78411 awarded by the National Institutes of Health. The Government has certain rights in the invention.

### FIELD OF THE INVENTION

The present invention relates to methods for determining the effectiveness of one or more agents, such as translation initiation inhibitors, for treating one or more disorders associated with aberrant cellular proliferation, and screening assays for the discovery of agents that alter eIF2α phosphorylation, inhibit translation initiation and/or inhibit aberrant cellular proliferation.

### BACKGROUND OF THE INVENTION

Translation, the mRNA-directed synthesis of proteins, occurs in three distinct steps: initiation, elongation and termination. Translation initiation is a complex process in which the two ribosomal subunits and methionyl tRNA (mtRNA) assemble on a properly aligned mRNA to commence chain elongation at the AUG initiation codon. The established scanning mechanism for initiation involves the formation of a ternary complex among eukaryotic initiation factor 2 (eIF2), GTP and met-tRNA. The ternary complex recruits the 40S ribosomal subunit to form the 43S pre-initiation complex. This complex recruits mRNA in cooperation with other initiation factors such as eIF4E, which recognizes the 7-methyl-guanidine cap (m-7GTP cap) in an mRNA molecule and forms the 48S pre-initiation complex. Cap recognition facilitates the 43S complex entry at the 5′ end of a capped mRNA. Subsequently, this complex migrates linearly until it reaches the first AUG codon, where a 60S ribosomal subunit joins the complex, and the first peptide bond is formed (Pain (1996) Eur. J. Biochem. 236:747-771).

Translation initiation plays a critical role in the regulation of cell growth and malignant transformation because expression of most oncogenic and cell growth regulatory proteins is translationally regulated (Flynn et al. (1996) Cancer Surv. 27:293; Sonenberg et al. (1998) Curr. Opin. Cell Biol. 10:268). For this reason, translation initiation is a tightly regulated cellular process. Many examples demonstrate that disregulation of translation initiation contributes to the genesis and progression of cancer (Donze et al. (1995) Embo J. 14: 3828; Rosenwald (1996) Bioessays 18: 243-50. (1996); De Benedetti et al. (2004) Oncogene 23: 3189-99 (2004); and Rosenwald (2004) Oncogene 23:3230). Conversely, inhibition of translation initiation reverts transformed phenotypes (Jiang et al. (2003) Cancer Cell Int. 3:2; Graff et al. (1995) Int. J. Cancer 60:255). The eIF2GTPMet-tRNAi complex is a critical site in the regulation of translation initiation that is “targeted” by novel anti-cancer agents generally known as inhibitors of translation initiation.

Following the recruitment of the 60S ribosomal subunit at the AUG initiator codon, the GTP bound to eIF2 in the ternary complex is hydrolyzed to GDP. This GDP must be exchanged for another molecule of GTP in order to regenerate the ternary complex and initiate a new round of translation. This GDP-GTP exchange is catalyzed by the guanine nucleotide exchange factor eIF2B, and is inhibited when eIF2α is phosphorylated. Phosphorylated eIF2α has a much higher affinity for and inhibits the function of eIF2B because when bound to phosphorylated eIF2α, eIF2B cannot catalyze the GDP-GTP exchange (Pain (1996) Eur. J. Biochem. 236:747). In other words, phosphorylated eIF2α is an inhibitor of eIF2B. Because the stoichiometric ratio of eIF2B and eIF2α in the cytosol is quite low, i.e., molecules of eIF2α are far more abundant than molecules of eIF2B, even partial phosphorylation of eIF2α is sufficient to decrease the availability of eIF2B necessary to re-cycle the eIF2.GDP into the functional eIF2.GTP form. The net result of even partial phosphorylation of eIF2α is a reduction in the availability of the ternary complex necessary to initiate a new round of translation (Brostrom et el. (1989) J Biol. Chem. 264:1644; Srivastava et al. (1995) J Biol. Chem. 270:16619).

eIF2α is phosphorylated on its serine 51 residue by eIF2α protein kinases including PKR (protein kinase R) and PERK (PKR-like protein kinase). eIF2α kinases are activated by signals from a “stressed” endoplasmic reticulum triggering a cascade of events generally termed the endoplasmic reticulum (ER) stress response. Most proteins synthesized in the cytoplasm are translocated to the ER for folding and post-translational modifications. Increased protein synthesis that overwhelms the ER's capacity for folding or other disturbances that prevent protein folding or transport, induce ER stress (Harding et al. (2000) Mol. Cell 5:897; Kaufman (1999) Genes Dev. 13:1211). Another ER stressor is the partial depletion of intracellular Ca⁺⁻ stores, probably because Ca⁺⁺ stored in the ER contributes to protein folding, although the exact mechanism by which reduction of ER calcium triggers the ER stress response is not clearly understood. It is well established and supported by an extensive body of experimental evidence that partial depletion of Ca⁺⁺ stores rapidly induces the ER stress response, and activates eIF2α kinases phosphorylating eIF2α and reducing the rate of translation initiation (Brostrom et al. (1989) J. Biol. Chem. 264:1644; Aktas et al. (1998) Proc. Natl. Acad. Sci. U.S.A. 95 :8280).

Phosphorylation of eIF2α limits the availability of the ternary complex and preferentially affects the translation of mRNAs coding for oncogenic proteins such as the GI cyclins or c-myc but not or much less the translation of housekeeping proteins such as β-actin or ubiquitin (Clemens et al. (1999) Int. J. Biochem. Cell Biol. 31:1). Paradoxically, a subset of mRNAs is translated more efficiently when the ternary complex is scarce than when it is abundant (Aktas et al. (2004) Journal of Nutrition in press). These include the mRNA encoding for the transcription factor ATF-4, which transcriptionally up-regulates many of the ER stress response genes such as pro-apoptotic C/EBP-homologous protein (CHOP) or the ER chaperone binding protein (BiP) (Harding et al. (2000) Mol. Cell 6:1099). An isoform of the BRCA1 mRNA belongs to this subset of mRNAs that are more efficiently translated when the ternary complex is scarce. Ca⁺⁺-depleting inhibitors of translation initiation up-regulate CHOP and BiP in cancer cells and in tumors excised from either animal cancer models or human patients. These compounds also increase the translation of BRCA1 mRNAb in breast cancer cell lines.

### SUMMARY OF THE INVENTION

Regulation of translation initiation plays a critical role in the control of cell proliferation, apoptosis and cancer because expression of growth regulatory, pro-apoptotic, oncogenic, and tumor suppressor proteins is tightly regulated at the level of translation and is highly dependent on the activity of translation initiation factors. In many human cancers, some degree of disregulation of translation initiation favors the expression of oncogenic over “house-keeping” proteins and contributes to the initiation and maintenance of the malignant phenotypes. Without intending to be bound by theory, inhibitors of translation initiation such as n-3 polyunsaturated fatty acid eicosapentaenoic acid (EPA), troglitazone (TRO) or clotrimazole (CLT) restore translational control to reduce the expression of oncogenic proteins, and favor the expression of pro-apoptotic proteins and tumor-suppressor proteins, thereby suppressing malignant phenotypes. Accordingly, translation initiation represents an attractive target for treatment and/or prevention of cancer, and for the development of new translation initiation inhibitors for anti-cancer therapy that induce phosphorylation eIF2α and thereby restrict the availability of the ternary complex in human cancers.

As used herein the term “target accreditation” is intended to include, but is not limited to, the demonstration that mechanism-specific anti-cancer agents affect the same target in vivo that they affect in vitro. One powerful accreditation strategy is to mutate the putative target in a manner that ablates its response to the test agent rendering cancers with this mutation resistant to treatment. This can only be achieved in animal models, however.

Another tool for target accreditation is the identification of biomarkers as reporters of target-specific activity of the drug in vivo, ideally in humans. However, very few if any anti-cancer drugs currently in use for the treatment of human cancers met the accreditation criterion in humans, i.e. the demonstration that a given “target-specific drug” does indeed interact with its putative target in the human cancers against which the drug is used.

The present invention is based on the discovery that eIF2α phosphorylation is up-regulated by certain inhibitors of translation initiation that are effective for reducing aberrant cellular proliferation. Accordingly, embodiments of the present invention are directed to novel methods for identifying agents that inhibit aberrant cellular proliferation in a cell. The methods include determining eIF2α phosphorylation in the cell (step 1), contacting the cell with an agent, determining the level of eIF2α phosphorylation in the cell (step 2), and comparing the level of eIF2α phosphorylation in the first step with the level of eIF2α phosphorylation in the second step. If the level of eIF2α phosphorylation in step 2 is greater than the level of eIF2α phosphorylation in step 1, the agent inhibits aberrant cellular proliferation.

In one aspect, a disorder associated with aberrant cellular proliferation is cancer. In another aspect, an agent for treating one or more disorders associated with aberrant cellular proliferation is an agent that inhibits translation initiation, and/or activates an eIF2α kinase. In certain aspects, the cell is obtained from a biological sample and/or from a mammal. In other aspects, eIF2α phosphorylation is detected with a phospho-specific anti-eIF2α antibody. In certain aspects, an agent is an agonist, antagonist, peptidomimetic, protein, peptide, nucleic acid or small molecule.

Embodiments of the present invention are further directed to methods for monitoring the effectiveness of an agent for treating a disorder associated with aberrant cellular proliferation in a biological sample. The methods include determining a level of eIF2α phosphorylation in a biological sample (step 1), contacting the biological sample with the agent, determining the level of eIF2α phosphorylation in the biological sample (step 2), and comparing the level of eIF2α phosphorylation in step 1 with the level of eIF2α phosphorylation in step 2. If the level of eIF2α phosphorylation in step 2 is greater than the level of eIF2α phosphorylation in step 1, the agent effectively treats the disorder associated with aberrant cellular proliferation.

In one aspect, a disorder associated with aberrant cellular proliferation is cancer. In another aspect, an agent for treating one or more disorders associated with aberrant cellular proliferation is an agent that inhibits translation initiation, and/or activates an eIF2α kinase. In certain aspects, a biological sample is from an organism and may be a tissue culture sample, a cell or a tissue sample. Biological samples may be obtained from an animal model of aberrant cellular proliferation or from a subject suffering from a disorder associated with aberrant cellular proliferation. In other aspects, the biological sample is contacted with two or more agents.

Embodiments of the present invention are further directed to methods for monitoring the effectiveness of an agent for treating a disorder associated with aberrant cellular proliferation in a subject in need thereof. The methods include obtaining a first biological sample from the subject, determining the level of eIF2α phosphorylation in the first biological sample, administering an agent to the subject, obtaining a second biological sample from the subject, determining the level of eIF2α phosphorylation in the second biological sample, and comparing the level of eIF2α phosphorylation in the first biological sample with the level of eIF2α phosphorylation in the second biological sample. If the level of eIF2α phosphorylation in the second biological sample is greater than the level of eIF2α phosphorylation in the first biological sample, the agent effectively treats the disorder associated with aberrant cellular proliferation in the subject in need thereof. The amount of agent administered may be altered to increase or decrease the level of eIF2α phosphorylation in the second biological sample.

In certain aspects, the first and second biological samples can be biological fluid samples, tissue samples and/or cell samples. In other aspects, the subject is a mammal. In yet other aspects, the aberrant cellular proliferation is cancer such as prostate cancer, breast cancer and ovarian cancer. In certain aspects, the agent inhibits translation initiation, activates an eIF2α kinase, inhibits tumor growth, inhibits tumor metastasis, and/or increases the life expectancy of the subject.

Embodiments of the present invention are further directed to methods of quantifying aberrant cellular proliferation. The methods include obtaining a test sample, determining the level of eIF2α phosphorylation in the test sample, obtaining a standard sample, determining the level of eIF2α phosphorylation in the standard sample, and comparing the level of eIF2α phosphorylation in the test sample and the level of eIF2α phosphorylation in the standard sample. The degree to which eIF2α phosphorylation in the test sample is greater than the level of eIF2α phosphorylation in the standard corresponds to the degree of aberrant cellular proliferation in the test sample.

Embodiments of the present invention are also directed to kits for determining aberrant cellular proliferation in a sample. The kits may include a compound that detects phosphorylated eIF2α, a standard sample and/or instructions for use.

### BRIEF DESCRIPTION OF THE DRAWINGS

The patent or application file contains at least one drawing executed in color. Copies of this patent or patent application publication with color drawings will be provided by the Office upon request and payment of the necessary fee. The foregoing and other features and advantages of the present invention will be more fully understood from the following detailed description of illustrative embodiments taken in conjunction with the accompanying drawings in which:

FIGS. 1A-1D depict representative photographs of a prostate biopsy (left, A and C) and a radical prostatectomy specimen (right, B and D) from one of two prostate cancer patients treated with 15 grams of OMEGARX™ fish oil for 5 weeks prior to radical prostatectomy (top, A and B), and from one of two prostate cancer patients treated for the same time period with 15 grams of corn oil (bottom, C and D). Both biopsy and prostatectomy specimens were immunostained with anti-P-51S-eIF2α, a phospho-specific antibody. Staining with anti-total eIF2α antibodies did not show any difference between biopsy and prostatectomy samples from subjects treated with either fish or corn oil.

FIGS. 2A-2E depicts the specificity of anti-S51-P-eIF2α antisera. (A) Anti-S51-P-eIF2α stained prostate tissue. (B) Same as A but with antibody pre-absorbed with excess antigenic peptide used for raising the antibody. (C) Staining of serial sections to A and B with anti-total eIF2α. (D) Staining of prostate tissue with anti-BiP antibodies. (E) Staining of primary breast cancer with anti-cyclin D1 antibody.

FIG. 3 depicts phosphorylation of eIF2α (left) and expression of BiP (right) induced by oral administration of TRO to liposarcoma patients between biopsy and surgery. Pre-TRO is a biopsy sample; Post-TRO is a sample obtained by surgery.

FIGS. 4A-4C depict dose-dependent up-regulation of BRCA1 in MCF-7 breast cancer cells by EPA (A) and CLT (B). Upper bands, BRCA1; lower bands, actin. (C) graphically depicts a CLT-induced increase of firefly luciferase expression when its mRNA contains the 5′ UTR of BRCA1 mRNAb (gray) but not the 5′ UTR of BRCA1 mRNAa (black).

FIGS. 5A-5B depict increases in the EPA content of RBC membranes in response to administration of OMEGARX™ fish oil. (A) Graphical representation of EPA content (relative to a standard) of the RBC membranes in three subjects before (0) and after (5) starting either fish oil (gray) or corn oil (black) intake. (B) Graphical representation of results from a study in which volunteers that took ten grams of either fish oil or corn oil for three weeks. The graph depicts the percent change in EPA in red blood cell (RBC) membranes after three weeks (mean±SEM).

FIGS. 6A-6B depict detection of phosphorylated eIF2α and BiP in human subjects. (A) Oral administration of TRO to liposarcoma patients after biopsy and prior to surgery induced phosphorylation of eIF2α (left) and expression of BiP (right). Pre is the biopsy sample; Post is the sample obtained from surgery. (B) Scores of samples from four patients (provided by author of Berridge (1995) Biochem J 312:1).

FIG. 7 depicts the effect of EPA on intracellular calcium. EPA induces ER calcium depletion in cells transfected with Ca⁻⁺ sensitive ER-targeted chameleon proteins.

FIGS. 8A-8C depict EPA activation of eIF2α kinases, phosphorylation of eIF2α, and inhibition of translation initiation in DU-145 prostate cancer cells. (A) and (B) lysates of EPA-treated cells were blotted with antibodies to phosphorylated (active) PERK or phosphorylated eIF2α. HSP90 or total eIF2α were used for loading control. Upper bands, phosphorylated eIF2α; lower bands, total eIF2α. (C) depicts a polysome profile of EPA-treated human prostate cancer cells. Lysates of EPA-treated cells were separated by sucrose density gradient centrifugation. Gradients were eluted under monitoring at 254 nm.

FIG. 9 depicts cell cycle inhibition by EPA that is dependent on eIF2α phosphorylation. Cancer cells transfected with either eIF2α-WT or eIF2α-51A and then exposed to 30 μM EPA for 2 hours. eIF2α phosphorylation was measured by Western blot.

FIG. 10 depicts cell cycle inhibition by EPA that is dependent on eIF2α phosphorylation. DU-145 human prostate cancer cell lines were transiently transfected with eIF2α-51A-GFP or eIF2α-WT-GFP plasmids, treated with EPA or vehicle, stained with propidium iodide and cell cycle distribution of GFP transfected cells was determined. The FL1-H channel shows GFP intensity while the FL2-A channel shows propidium iodide intensity.

FIG. 11 depicts EPA down-regulation of cyclin D1 in DU-145 prostate cancer cells, as determined by Western blot. β-actin is used as a loading control.

FIG. 12 depicts EPA regulation of oncogenes. Upper panel: Exponentially growing NIH 3T3 cells were pulsed with [³⁵S]met-cys with or without 30 μM EPA for one hour.

The cells were lysed and cell lysates were immunoprecipitated with anti-cyclin D1, -cyclin E, -Ras, -β-actin or -ubiquitin antibodies, the immunoprecipitates were analyzed by SDS-PAGE and developed by autoradiography. Lower panel: Western blot analysis of EPA-treated cells after six hours.

FIGS. 13A-13B depict EPA induction of BiP and CHOP expression in DU-145 cells. (A) Western blot analysis of BiP and CHOP expression in EPA treated cells. (B) Graphical representation of the ratio of firefly to renilla luciferase activity normalized to the ratio in vehicle. DU-145 cells were transiently transfected with plasmids coding for either firefly luciferase (L) preceded by the 5′ UTR of ATF-4 or for renilla luciferase (R) preceded by a simple 5′ UTR. Expression of reporters was detected by dual luciferase assay after treatment with vehicle or EPA.

FIGS. 14A-14F depict the effect of EPA on xenograft cancer models. (A) EPA increased the mean survival time in an orthotopic model of PC-3 human prostate tumors (left, corn oil; right, EPA). (B) EPA reduced tumor growth in an orthotopic syngenic model of KLN mouse squamous cell carcinoma (5 weeks) (left, corn oil; right, EPA). (C) EPA induced phosphorylation of eIF2α in the KLN tumors (left, corn oil; middle, EPA1;

right, EPA2). EPA1 and EPA2 are samples stained with the same antibody four years apart. (D) EPA reduced expression of cyclin D1 in the KLN tumors (left, corn oil; right, EPA). (E) Specificity of the anti-S51-P-eIF2α antibody in cells treated without (−) or with (+) thapsigargin (TG) to induce eIF2α phosphorylation. Top band is phosphorylated eIF2α; bottom band is eIF2α total. (F) EPA increased expression of BiP in the KLN tumors (left, corn oil; right, EPA). (C, D, and F are immunostainings from tumors in B).

FIG. 15 depicts the effect of EPA on p53^{-/-} mice. p53^{-/-} mice were started on oral EPA treatment (2.5 grams/kilogram/day) or vehicle (corn oil) at four weeks of age and continued until the death of the last animal. The Kaplan-Meier survival analysis of this experiment revealed a mean survival time of 196±9 days for the vehicle-control animals, and of 385±12 days (p<0.005; confidence 0.995%) for the EPA-treated mice.

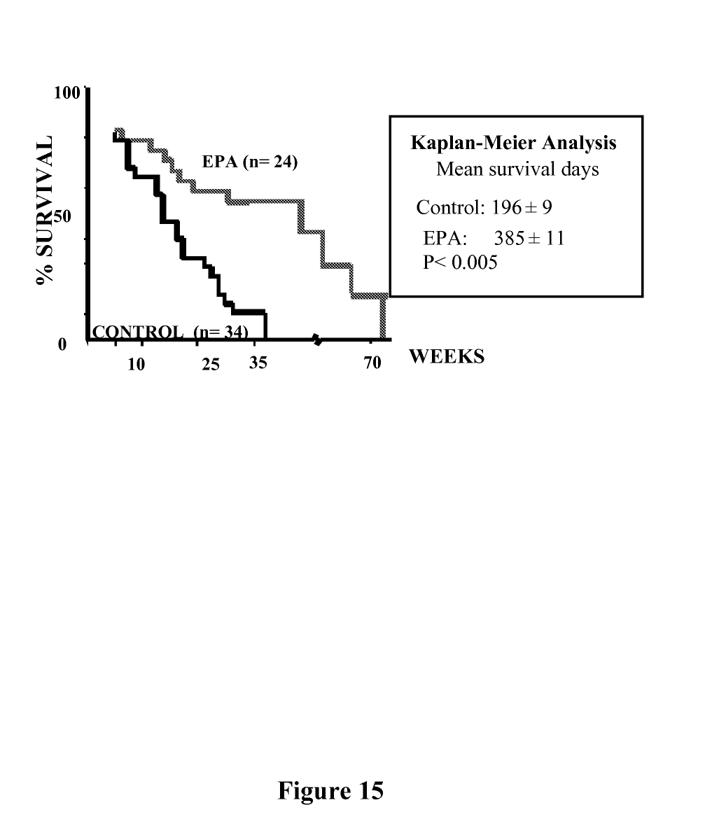

### DETAILED DESCRIPTION OF THE INVENTION

Embodiments of the present invention are based on the discovery that eIF2α phosphorylation may be used as a prognostic tool for determining the effectiveness of inhibitors of translation initiation in the treatment of disorders associated with aberrant cellular proliferation, such as cancer. Further embodiments of the present invention are directed to methods of screening for agents that promote eIF2α phosphorylation. In one aspect, agents that promote eIF2α phosphorylation also inhibit translation initiation. In another aspect, agents that promote eIF2α phosphorylation are effective for treating one or more disorders associated with aberrant cellular proliferation.

In one aspect, the present invention is directed to determining the ability of one or more of the agents described herein to treat one or more disorders associated with aberrant cellular proliferation, such as cancer. Treating a disorder associated with aberrant cellular proliferation is intended to include, but is not limited to, inhibition of proliferation including rapid proliferation. As used herein, the term “disorder associated with aberrant cellular proliferation” includes, but is not limited to, disorders characterized by undesirable or inappropriate proliferation of one or more subset(s) of cells in a multicellular organism. The term “cancer” refers to various types of malignant neoplasms, most of which can invade surrounding tissues, and may metastasize to different sites (PDR Medical Dictionary 1st edition (1995)). The terms “neoplasm” and “tumor” refer to an abnormal tissue that grows by cellular proliferation more rapidly than normal and continues to grow after the stimuli that initiated proliferation is removed (PDR Medical Dictionary 1st edition (1995)). Such abnormal tissue shows partial or complete lack of structural organization and functional coordination with the normal tissue which may be either benign (i.e., benign tumor) or malignant (i.e., malignant tumor).

The language “treating a disorder associated with aberrant cellular proliferation” is intended to include the prevention of the growth of neoplasms in a subject or a reduction in the growth of pre-existing neoplasms in a subject. The inhibition also can be the inhibition of the metastasis of a neoplasm from one site to another. In one aspect, the neoplasms are sensitive to one or more translation initiation inhibitors described herein. Examples of the types of neoplasms intended to be encompassed by the present invention include but are not limited to those neoplasms associated with cancers of the breast, skin, bone, prostate, ovaries, uterus, cervix, liver, lung, brain, larynx, gallbladder, pancreas, rectum, parathyroid, thyroid, adrenal gland, immune system, neural tissue, head and neck, colon, stomach, bronchi, and/or kidneys.

Treatment of one or more disorders associated with aberrant cellular proliferation includes reducing aberrant cellular proliferation such that the aberrant proliferation is reduced or eliminated or remission is obtained. Aberrant cellular proliferation can be reduced by approximately 1%, 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99% or 100%. Treatment also includes increasing the life expectancy of an organism (i.e., any of the organisms described herein) such that the organism with aberrant cellular proliferation will survive longer than it would if it were not subjected to treatment. For example, the life expectancy of an organism with aberrant cellular proliferation receiving treatment may be increased as compared to an organism with aberrant cellular proliferation not receiving treatment. Life expectancy can be increased by hours (e.g., in an organism such a C. elegans or D. melanogaster), days, weeks, months or years.

Prognostic Assays

The prognostic assays described herein can be used to determine whether a subject can be administered an agent (e.g., an agonist, antagonist, peptidomimetic, protein, peptide, nucleic acid, small molecule, or other drug candidate) to promote the phosphorylation of eIF2α. In one aspect, an agent that promotes the phosphorylation of eIF2α is used to treat a disorder associated with aberrant cellular proliferation. Thus, the prognostic assays described herein may be used to determine whether a subject can be effectively treated with one or more agents to alleviate or reduce one or more disorders associated with aberrant cellular proliferation.

In one embodiment, the present invention provides a method for determining and/or monitoring the effectiveness of treatment of a subject with an agent (e.g., an agonist, antagonist, peptidomimetic, protein, peptide, nucleic acid, small molecule, or other drug candidate) including the steps of obtaining a pre-administration sample from a subject prior to administration of the agent; detecting eIF2α phosphorylation in the pre-administration sample; obtaining one or more post-administration samples from the subject; detecting the level of eIF2α phosphorylation in the post-administration samples; comparing the level of eIF2α phosphorylation in the pre-administration sample with the level of eIF2α phosphorylation post administration sample or samples; and altering the administration of the agent to the subject accordingly. For example, increased administration of the agent may be desirable to increase eIF2α phosphorylation to higher levels than detected, i.e., to increase the effectiveness of the agent. Alternatively, decreased administration of the agent may be desirable to decrease eIF2α phosphorylation to lower levels than detected, i.e., to decrease the effectiveness of the agent. According to such an embodiment, eIF2α phosphorylation may be used as an indicator of the effectiveness of an agent, even in the absence of an observable phenotypic response.

In another embodiment, the methods of the invention involve obtaining a control biological sample from a control subject, contacting the control sample with one or more of the agents described herein, and comparing the reactivity (e.g., eIF2α phosphorylation) of the agent to the control sample with the reactivity of the agents (e.g., eIF2α phosphorylation) to a test sample. In another embodiment, the level of eIF2α phosphorylation is used quantify the level of aberrant cellular proliferation and/or translation initiation, as well as to follow the progression and/or regression of a disorder associated with aberrant cellular proliferation.

In certain aspects of the invention, an agent alters eIF2α phosphorylation, translation initiation and/or aberrant cellular proliferation (e.g., in a cell, tissue, fluid or organism).

For example, in the presence of the agent, eIF2α phosphorylation, translation initiation and/or aberrant cellular proliferation levels may be increased to levels that are higher than levels that occur in the absence of the agent. eIF2α phosphorylation, translation initiation and/or aberrant cellular proliferation levels may be increased by about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 600%, 700%, 800%, 900%, 1000% or more relative to levels of eIF2α phosphorylation, translation initiation and/or aberrant cellular proliferation in the absence of the agent.

eIF2α phosphorylation, translation initiation and/or aberrant cellular proliferation levels may also be decreased (e.g., inhibited) in the presence of an agent to levels that are lower than levels that occur in the absence of the agent. For example, eIF2α phosphorylation, translation initiation and/or aberrant cellular proliferation levels may be decreased by about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 225%, 250%, 275%, 300%, 325%, 350%, 375%, 400%, 425%, 450%, 475%, 500%, 600%, 700%, 800%, 900%, 1000% or more relative to levels of eIF2α phosphorylation, translation initiation and/or aberrant cellular proliferation in the absence of the agent.

As used herein, a “test sample” refers to a biological sample obtained from a subject of interest. For example, a test sample can be a biological fluid sample (e.g., serum, sputum, urine), tissue sample (e.g., a biopsy) or cell sample (e.g., a cheek scraping). As used herein, a “normal sample” or a “standard sample” refers to a biological sample obtained from a healthy (i.e., non-malignant) biological fluid sample, tissue sample or cell sample. As used herein, the term “biological sample” is intended to include tissues, cells and biological fluids isolated from a subject, as well as tissues, cells and fluids present within a subject. Biological samples may be of any biological tissue or fluid or cells. Typical biological samples include, but are not limited to, sputum, lymph, blood, blood cells (e.g., white cells), fat cells, cervical cells, cheek cells, throat cells, mammary cells, muscle cells, skin cells, liver cells, spinal cells, bone marrow cells, tissue (e.g., muscle tissue, cervical tissue, skin tissue, spinal tissue, liver tissue and the like) fine needle biopsy samples, urine, cerebrospinal fluid, peritoneal fluid and pleural fluid, or cells therefrom. Biological samples may also include sections of tissues such as frozen sections taken for histological purposes. A biological sample may be obtained from a mammal, including, but not limited to horses, cows, sheep, pigs, goats, rabbits, guinea pigs, rats, mice, gerbils, non-human primates and humans. Biological samples may also include cells from microorganisms (e.g., bacterial cells, viral cells, yeast cells and the like) and portions thereof. As used herein, the term “biological fluid” is intended to include any fluid taken from a biological organism. Biological fluids include, but are not limited to, sputum, lymph, blood, urine, tears, breast milk, nipple aspirate fluid, seminal fluid, vaginal secretions, cerebrospinal fluid, peritoneal fluid, pleural fluid, pus, ascites and the like.

Monitoring the influence of agents (e.g., drugs) on the level of eIF2α phosphorylation in a sample can be applied in clinical trials. For example, the effectiveness of an agent (i.e., an agent such as EPA or an agent determined by a screening assay as described below) to increase eIF2α phosphorylation can be monitored in clinical trials of subjects exhibiting aberrant cellular proliferation.

Screening Assays

The present invention provides a method (also referred to herein as a “screening assay”) for identifying agents (e.g., peptides, cyclic peptides, peptidomimetics, small molecules, small organic molecules, or other drugs) which have a stimulatory or inhibitory effect on eIF2α phosphorylation, translation initiation and/or cell proliferation.

In one embodiment, the invention provides assays for screening candidate or test agents which phosphorylate eIF2α. The test agents of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries and natural product collections, while the other four approaches are applicable to peptide, non-peptide oligomer or small molecule libraries of compounds (Lam, K. S. (1997) Anticancer Drug Des. 12: 145).

As used herein, the term “small organic molecule” refers to an organic molecule, either naturally occurring or synthetic, that has a molecular weight of more than about 25 daltons and less than about 3000 daltons, preferably less than about 2500 daltons, more preferably less than about 2000 daltons, preferably between about 100 to about 1000 daltons, more preferably between about 200 to about 500 daltons.

The agents of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented in solution (e.g., Houghten (1992) Biotechniques 13:412), or on beads (Lam (1991) Nature 354:82), chips (Fodor (1993) Nature 364:555), bacteria (Ladner U.S. Pat. No. 5,223,409), spores (Ladner U.S. Pat. No. 5,223,409), plasmids (Cull et al. (1992) Proc. Natl. Acad. Sci. USA 89:1865) or on phage (Scott and Smith (1990) Science 249:386); (Devlin (1990) Science 249:404); (Cwirla et al. (1990) Proc. Natl. Acad. Sci. USA 87:6378); (Felici (1991) J. Mol. Biol. 222:301); (Ladner supra).

Examples of methods for introducing a molecular library of randomized nucleic acids into a population of cells can be found in the art, for example in U.S. Pat. No. 6,365,344, incorporated herein in its entirety by reference. A molecular library of randomized nucleic acids can provide for the direct selection of candidate or test agents with desired phenotypic effects. The general method can involve, for instance, expressing a molecular library of randomized nucleic acids in a plurality of cells, each of the nucleic acids comprising a different nucleotide sequence, screening for a cell of exhibiting a changed physiology in response to the presence in the cell of a candidate or test agent, and detecting and isolating the cell and/or candidate or test agent.

In one embodiment, the introduced nucleic acids are randomized and expressed in the cells as a library of isolated randomized expression products, which may be nucleic acids, such as mRNA, antisense RNA, siRNA, ribozyme components, etc., or peptides (e.g., cyclic peptides). The library should provide a sufficiently structurally diverse population of randomized expression products to effect a probabilistically sufficient range of cellular responses to provide one or more cells exhibiting a desired response. Generally at least 10⁶, at least 10⁷, at least 10⁸, or at least 10⁹ different expression products are simultaneously analyzed in the subject methods. In one aspect methods maximize library size and diversity.

The introduced nucleic acids and resultant expression products are randomized, meaning that each nucleic acid and peptide consists of essentially random nucleotides and amino acids, respectively. The library may be fully random or biased, e.g., in nucleotide/residue frequency generally or per position. In other embodiments, the nucleotides or residues are randomized within a defined class, e.g. of hydrophobic amino acids, of purines, etc.

Functional and structural isolation of the randomized expression products may be accomplished by providing free (not covalently coupled) expression product, though in some situations, the expression product may be coupled to a functional group or fusion partner, such as a heterologous (to the host cell) or synthetic (not native to any cell) functional group or fusion partner. Exemplary groups or partners include, but are not limited to, signal sequences capable of constitutively localizing the expression product to certain tissues (e.g., prostate tissue, breast tissue, skin tissue and the like) or to a predetermined subcellular locale such as the Golgi, endoplasmic reticulum, nucleoli, nucleus, nuclear membrane, mitochondria, chloroplast, secretory vesicles, lysosome, and the like; binding sequences capable of binding the expression product to a predetermined protein while retaining bioactivity of the expression product; sequences signaling selective degradation, of itself or co-bound proteins; and secretory and membrane-anchoring signals.

In one embodiment, an assay is a cell-based assay in which one or more of a normal cell, a precancerous cell and/or a cancer cell is contacted with an agent and the ability of the agent to increase or decrease eIF2α phosphorylation is determined in a standard sample and a test sample including the agent. In one aspect, an increase in eIF2α phosphorylation indicates that the test agent is an inhibitor of translation initiation. In another aspect, an increase in eIF2α phosphorylation indicates that the test agent may be used to treat one or more disorders associated with aberrant cellular proliferation.

One agent for detecting phosphorylated eIF2α is an antibody capable of binding to eIF2α or a portion thereof, such as an antibody with a detectable label. In one aspect, the antibody only binds to phosphorylated eIF2α, i.e., eIF2α phosphorylated at serine 51. Antibodies which bind only to phosphorylated eIF2α are described herein. Antibodies can be polyclonal or monoclonal. An intact antibody, or a fragment thereof (e.g., Fab or F(ab′)₂) can be used. The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The detection method of the invention can be used, for example, to detect phosphorylated eIF2α in a biological sample in vitro as well as in vivo. For example, in vitro techniques for detection of phosphorylated eIF2α include enzyme linked immunosorbent assays (ELISAs), Western blots, immunoprecipitations and immunofluorescence. In vivo techniques for detection of phosphorylated eIF2α include introducing into a subject a labeled anti-eIF2α antibody, such as a labeled anti-eIF2α antibody specific for eIF2α phosphorylated at serine 51. For example, the antibody can be labeled with a radioactive marker whose presence and location in a subject can be detected by standard imaging techniques.

In another embodiment, eIF2α can be labeled with ³²P, either directly or indirectly, and the radioisotope detected by direct counting of radioemmission or by scintillation counting. Alternatively, eIF2α may be coupled with ³⁵S, ³²P, ¹⁴C, or ³H, either directly or indirectly, and the radioisotope detected by direct counting of radioemmission or by scintillation counting. Alternatively, substances can be enzymatically labeled with, for example, horseradish peroxidase, alkaline phosphatase, or luciferase, and the enzymatic label detected by determination of conversion of an appropriate substrate to product. Alternatively, it is also within the scope of this invention to determine the ability of an agent of the invention to interact with a sample without the labeling of any of the interactants.

In one embodiment, it may be desirable to immobilize the agent (e.g., an compound such as a phospho-specific antibody) to facilitate separation of phosphorylated eIF2α from unphosphorylated eIF2α, as well as to accommodate automation of the assay. Interaction of an antibody with phosphorylated eIF2α in the presence and absence of a candidate agent, for example, can be accomplished in any vessel suitable for containing the reactants. Examples of such vessels include microtitre plates, test tubes and microfuge tubes. In one embodiment, the antibody can be adsorbed onto beads, such as magnetic beads, or derivatized microtitre plates, which are then combined with the agent and the sample, and the mixture incubated under conditions conducive to complex formation. Following incubation, the beads or microtitre plate wells are washed to remove any unbound components, the matrix immobilized in the case of beads, complex determined either directly or indirectly, for example, as described above. Alternatively, the complexes can be dissociated from the matrix, and the level eIF2α binding determined using standard techniques.

This invention further pertains to novel agents identified by the above-described screening assays. Accordingly, it is within the scope of this invention to further use an agent identified as described herein in an appropriate animal model. For example, an agent as described herein can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays in animal models to determine effective treatment parameters for disorders associated with aberrant cellular proliferation.

In one embodiment, an assay is an animal model based assay comprising contacting an animal with one or more test agents and determining the ability of the test agents to phosphorylate eIF2α. In one aspect, an agent that phosphorylates eIF2α is an inhibitor of translation initiation. In another aspect, an agent that phosphorylates eIF2α treats one or more disorders associated with aberrant cellular proliferation. In one aspect, the animal is an animal model of cancer. An animal model of cancer includes, but is not limited to, a xenograft cancer model wherein the animal expresses a human cancer. In one aspect, the animal is a mouse (e.g., a xenograft mouse such as a nude mouse). Animal cancer models including xenograft cancer models are known in the art and are described in Teicher, B.A., editor, Tumor Models in Cancer Research, Humana Press, 2002, incorporated herein in its entirety by reference.

As used herein, the term cell include cells obtained from samples derived directly from the animal models (e.g., wild-type, transgenic, and knockout) and human subjects described herein (e.g., biological samples), as well as cells which are derived from the samples described herein and have been grown in tissue culture. Cells may also include those obtained from established cell lines including tumor cell lines such as adenocarcinoma, Burkitt's lymphoma, colorectal carcinoma, ductal carcinoma, epidermoid carcinoma, Ewing's sarcoma, fibrosarcoma, gastric carcinoma, giant cell sarcoma, hepatocellular carcinoma, Hodgkin's disease, lymphoma, malignant melanoma, neuroblastoma, osteocarcinoima, plasmacytoma, primary ductal carcinoma, rhabdomyosarcoma, squamous cell carcinoma, transitional cell carcinoma, uterine sarcoma; and general mammalian cell lines such as CV-1, HeLa, and COS, e.g., COS-7.

Pharmaceutical Compositions

The agents described herein (i.e., agents that affect eIF2α phosphorylation) can be incorporated into pharmaceutical compositions suitable for administration. Such compositions typically comprise the agent(s) and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active agent, use thereof in the compositions is contemplated. Supplementary active agents can also be incorporated into the compositions.

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active agent in the required amount in an appropriate solvent with one or a combination of ingredients enumerated above, as required, followed by filtered sterilization. Generally, dispersions are prepared by incorporating the active agent into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active agent can be incorporated with excipients and used in the form of tablets, troches, or capsules. Oral compositions can also be prepared using a fluid carrier for use as a mouthwash, wherein the agent in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In one embodiment, the active agents are prepared with carriers that will protect the agent against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811, incorporated herein by reference in its entirety.

The invention also encompasses kits for determining the effectiveness of one or more agents for treating one or more disorders associated with aberrant cellular proliferation. For example, the kit can comprise one or more of the agents described herein; means for determining eIF2α phosphorylation; and means for comparing eIF2α phosphorylation of the sample with a standard. The agent can be packaged in a suitable container. The kit can further comprise instructions for using the kit to determine the ability of one or more agents for treating one or more disorders associated with aberrant proliferation. Kits according to the present invention include, but are not limited to, kits for use in a clinical environment (e.g., hospital, clinic, physician's office and the like), in pathology laboratories, in scientific research laboratories and the like.

Recombinant Expression Vectors and Host Cells

Another aspect of the invention pertains to vectors, such as expression vectors, containing a nucleic acid encoding, for instance, eIF2α (or a portion thereof). As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression in prokaryotic or eukaryotic cells, e.g., bacterial cells such as E. coli, insect cells (using baculovirus expression vectors), yeast cells, amphibian cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of polypeptides in prokaryotes is most often carried out in E. coli with vectors containing constitutive or inducible promoters directing the expression of either fusion or non-fusion proteins. Fusion vectors add a number of amino acids to a polypeptide encoded therein, usually to the amino terminus of the recombinant polypeptide. Such fusion vectors typically serve three purposes: 1) to increase expression of recombinant polypeptide; 2) to increase the solubility of the recombinant polypeptide; and 3) to aid in the purification of the recombinant polypeptide by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Purified fusion polypeptide can be utilized in translation initiation activity assays, or to generate antibodies specific for eIF2α, for example.

Examples of suitable inducible non-fusion E. coli expression vectors include pTrc (Amann et al., (1988) Gene 69:301-315) and pET lid (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a co-expressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20:211 1). Such alteration of nucleic acid sequences of the invention can be carried out by standard DNA synthesis techniques.

In another embodiment, the expression vector is a yeast expression vector. Examples of vectors for expression in yeast S. cerevisiae include pYepSecl (Baldari et al., (1987) Embo J. 6:229), pMFa (Kurjan and Herskowitz, (1982) Cell 30:933), pJRY88 (Schultz et al., (1987) Gene 54:113), pYES2 (Invitrogen Corporation, San Diego, Calif.), and picZ (InVitrogen Corp, San Diego, Calif.).

Alternatively, insect cells and baculovirus expression vectors can be used. Baculovirus vectors available for expression of proteins in cultured insect cells (e.g., Sf 9 cells) include the pAc series (Smith et al. (1983) Mol. Cell Biol. 3:2156) and the pVL series (Lucklow and Summers (1989) Virology 170:31).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187). When used in mammalian cells, the expression vector's control functions are often provided by viral regulatory elements. For example, commonly used promoters are derived from polyoma, adenovirus 2, cytomegalovirus and Simian virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268), lymphoid-specific promoters (Calame and Eaton (1988) Adv. Immunol. 43:235), in particular promoters of T cell receptors (Winoto and Baltimore (1989) EMBO J. 8:729) and immunoglobulins (Banerji et al. (1983) Cell 33:729; Queen and Baltimore (1983) Cell 33:741), neuron-specific promoters (e.g., the neurofilament promoter; Byrne and Ruddle (1989) Proc. Natl. Acad. Sci. U.S.A. 86:5473), pancreas-specific promoters (Edlund et al. (1985) Science 230:912), and mammary gland-specific promoters (e.g., milk whey promoter; U.S. Pat. No. 4,873,316 and European Application Publication No. 264,166). Developmentally regulated promoters are also encompassed, for example the murine hox promoters (Kessel and Gruss (1990) Science 249:374) and the α-fetoprotein promoter (Campes and Tilghman (1989) Genes Dev. 3:537).

In one embodiment, the present invention provides a nucleic acid molecule which is antisense to a nucleic acid molecule of interest. As used herein, the term “antisense” refers to a nucleic acid that interferes with the function of DNA and/or RNA and may result in suppression of expression of the RNA and/or DNA. The antisense nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof.

An antisense nucleic acid molecule can be delivered to a cell to express an exogenous nucleotide sequence, to inhibit, eliminate, augment, or alter expression of an endogenous nucleotide sequence, or to express a specific physiological characteristic not naturally associated with the cell. In one embodiment, the antisense nucleic acid is an antisense RNA, an interfering double stranded RNA (“dsRNA”) or a short interfering RNA (“siRNA”). Antisense RNA and/or siRNA may be introduced into one or more cells using the vectors described herein.

As used herein, the term “siRNA” refers to double-stranded RNA that is less than 30 bases and preferably 21-25 bases in length. siRNA may be prepared by any method known in the art. For a review, see Nishikura (2001) Cell 16:415. In one embodiment, single-stranded, gene-specific sense and antisense RNA oligomers with overhanging 3′ deoxynucleotides are prepared and purified. For example, two oligomers, can be annealed by heating to 94° C. for 2 minutes, cooling to 90° C. for 1 minute, and then cooling to 20° C. at a rate of 1° C. per minute. The siRNA can then be injected into an animal or delivered into a desired cell type using methods of nucleic acid delivery described herein.

Another aspect of the invention pertains to host cells into which a recombinant expression vector of the invention has been introduced, containing sequences which allow it to homologously recombine into a specific site of the host cell's genome. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, host cells can be bacterial cells such as E. coli, insect cells, yeast, Xenopus cells, or mammalian cells (such as Chinese hamster ovary cells (CHO), African green monkey kidney cells (COS), or fetal human cells (293T)). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding a detectable translation product or can be introduced on a separate vector. Cells stably transfected with the introduced nucleic acid can be identified by drug selection (e.g., cells that have incorporated the selectable marker gene will survive, while the other cells die).

The host cells described herein may be infected with one or more viruses, such as a DNA or RNA animal virus. As used herein, RNA viruses include, but are not limited to, virus families such as picornaviridae (e.g., polioviruses), reoviridae (e.g., rotaviruses), togaviridae (e.g., encephalitis viruses, yellow fever virus, rubella virus), orthomyxoviridae (e.g., influenza viruses), paramyxoviridae (e.g., respiratory syncytial virus, measles virus, mumps virus, parainfluenza virus), rhabdoviridae (e.g., rabies virus), coronaviridae, bunyaviridae, flaviviridae, filoviridae, arenaviridae, bunyaviridae, and retroviridae (e.g., human T-cell lymphotropic viruses (HTLV), human immunodeficiency viruses (HIV)). As used herein, DNA viruses include, but are not limited to, virus families such as papovaviridae (e.g., papilloma viruses), adenoviridae (e.g., adenovirus), herpesviridae (e.g., herpes simplex viruses), and poxviridae (e.g., variola viruses). In one embodiment, the viral infection is caused by hepatitis B virus, hepatitis C virus, and/or HIV.

A host cell of the invention, such as a prokaryotic or eukaryotic host cell in culture, can be used to produce (i.e., express) a protein, e.g., eIF2α. Accordingly, the invention further provides methods for producing a protein using the host cells of the invention. In one embodiment, the method comprises culturing the host cell of invention (into which a recombinant expression vector encoding a detectable translation product has been introduced) in a suitable medium such that a detectable translation product is produced. In another embodiment, the method further comprises isolating an eIF2α protein from the medium or the host cell.

The host cells of the invention can also be used to produce nonhuman transgenic animals. For example, in one embodiment, a host cell of the invention is a fertilized oocyte or an embryonic stem cell into which eIF2α-coding sequences have been introduced. Such host cells can then be used to create non-human transgenic animals in which exogenous eIF2α sequences have been introduced into their genome. Such animals are useful for studying evaluating modulators of eIF2α. As used herein, a “transgenic animal” is a non-human animal, preferably a mammal, more preferably a rodent such as a rabbit, ferret, guinea pig, hamster, rat or mouse, in which one or more of the cells of the animal includes a transgene. Other examples of transgenic animals include non-human primates, sheep, dogs, cats, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing an exogenous nucleic acid into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to a detectable translation product transgene to direct expression of a detectable translation product to particular cells. Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al., and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of a detectable translation product transgene in its genome and/or expression of detectable translation product mRNA in tissues or cells of the animals. A transgenic founder animal can then be used to breed additional animals carrying the transgene. Moreover, transgenic animals carrying a transgene encoding a detectable translation product can further be bred to other transgenic animals carrying other transgenes.

This invention is further illustrated by the following examples, which should not be construed as limiting. The contents of all references, patents and published patent applications cited throughout this application are hereby incorporated by reference.

### EXAMPLE I

### Target Accreditation of EPA in Human Cancer

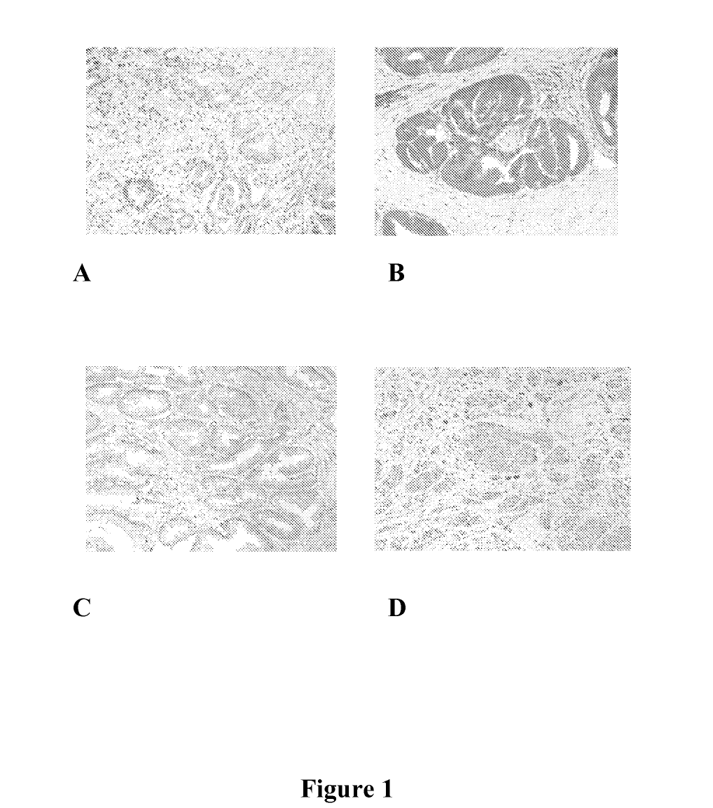

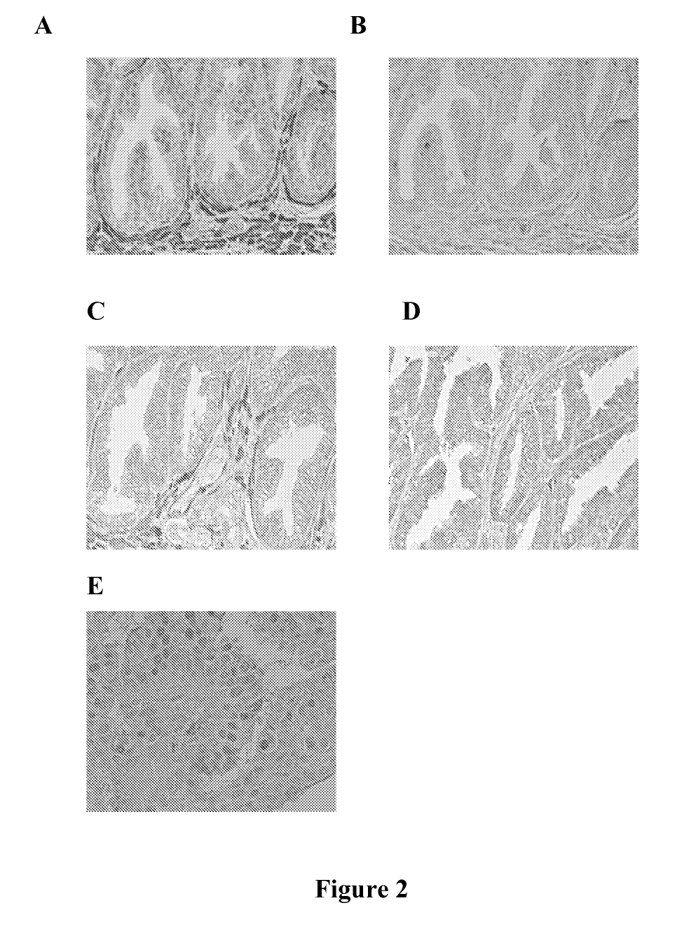

Using phospho-S51-specifc anti-eIF2α antibodies, it was determined that EPA and TRO, two types of Ca⁺⁺ mediated inhibitors of translation initiation, phosphorylated eIF2α tumors excised from cancer patients that were treated with one of these agents prior to surgery (FIG. 1A-1D and 2A-2E).

EPA

Although target accreditation is critical for the successful development of a target-specific anti-cancer drug, very few anti-cancer agents can be “accredited” in humans. Taking advantage of the fact that EPA is a food supplement, an Institutional Review Board (IRB) approved preliminary study was conducted in patients diagnosed with prostate cancer by prostate biopsy who later underwent radical prostatectomy at Brigham and Women's Hospital. In a double-blind, randomized, placebo-controlled study, the effect of each dietary intervention was assessed by comparing the level of eIF2α phosphorylation in the prostatectomy pathology specimens and in the patient's diagnostic biopsy. Two patients received 15 grams distilled purified fish oil (OMEGARX™, 75% EPA, 25% DHA, referred to hereafter as EPA) and two received 15 grams (equicaloric) of the n-6 polyunsaturated fatty acid (PUFA) corn oil, daily for five weeks prior to surgery. In both cases staining with anti-P-S51-eIF2α-specific antibody of a prostate biopsy sample (pre-oil intake) and of a radical prostatectomy sample (post-oil intake) was performed. In all cases, patients were administered the oil for five weeks prior to surgery and compliance was confirmed by fatty acid distribution in red blood membranes before and after the oil intake.

Levels of S51-P-eIF2α and total eIF2α were compared between the biopsy and the prostatectomy specimens of all subjects. In the prostatectomy specimens of the patients that received EPA prior to radical prostatectomy, there was a remarkable increase in phosphorylation of eIF2α as compared with the biopsy sample, although there was no change in the levels of total eIF2α that was not observed in the corn oil group. Both patients had similar results. FIGS. 1A-1B and 1C-1D each depict representative results from a single patient.

The specificity of the staining for phosphorylated eIF2α was determined by staining serial sections of prostate tissue with anti-S51-P-eIF2α before (FIG. 2A) and after (FIG. 2B) absorption of the antibody with excess amount of the phospho-peptide used as the immunogen to develop the anti-S5 1-P-eIF2α specific antibody. Staining in FIG. 2B also provides a negative control for the secondary antibody. Total eIF2α staining is depicted in FIG. 2C.

Up-regulation of BiP was detected in the prostate samples of the subjects that received EPA (FIG. 2D) but not in the controls. Up-regulation of BiP confirmed phosphorylation of eIF2α and restriction of the ternary complex. FIG. 2E depict staining of a primary breast cancer archival sample (paraffin-embedded) with anti-cyclin D1 antisera.

These data indicate that: 1) the dose of EPA used is adequate to induce changes in the phosphorylation level of eIF2α; 2) the phospho-eIF2α specific antibody was able to detect these changes by immunocytochemistry; and 3) that the changes in the phosphorylation levels of eIF2α observed in the patients that received fish oil did not likely result from non-specific factors related to the oil intake or the surgery as these changes were not observed in prostate samples from patients that received corn oil.

TRO

The anti-cancer effect of TRO has been shown to be mediated by Ca⁺⁺ release and phosphorylation of eIF2α in a manner comparable to that of EPA (Palakurthi, et al. (2001) Cancer Res. 61:6213). Pathology specimens were obtained from a clinical trial designed to assess the cell differentiation effect on human liposarcoma exerted by TRO administered between the time of biopsy and the time of surgery (Demetri et al. (1999) Proc. Natl. Acad. Sci. U.S.A. 96:3951). Samples were pre-treatment biopsies and post-treatment surgical specimens from the same patient, fixed and paraffin embedded.

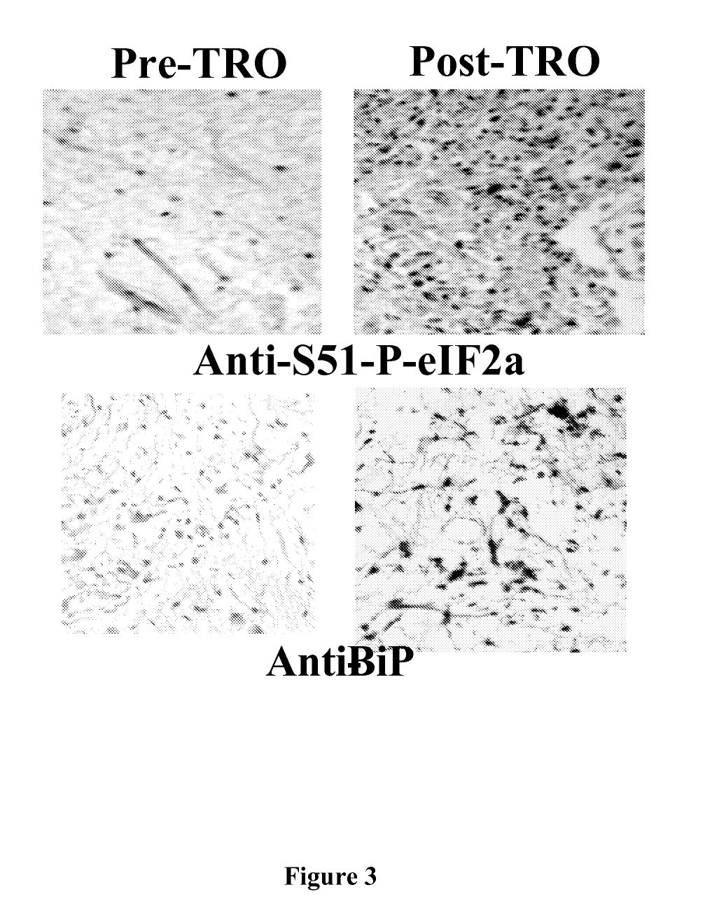

Analysis of these samples showed that oral administration of TRO caused significant phosphorylation of eIF2α and increase in BiP expression in post-treatment specimens as compared with the pre-treatment biopsy samples. FIG. 3 depicts one representative example. Three other subjects had similar results.

### EXAMPLE II

### Prevention of Recurrence or Spread of Metastases After Surgery for Treatment of Cancer

The present invention is also useful when a cancer patient is diagnosed by biopsy of having a particular type of cancer that is still amenable to surgical treatment. The patient will receive an inhibitor of translation initiation (e.g., EPA) for a period of time before surgery and then levels of eIF2α phosphorylation will be compared in the biopsy sample (i.e., a pre-surgical sample) to levels of eIF2α phosphorylation in the surgical samples.

An increase in eIF2α phosphorylation will support the continuous use of such a drug for prevention of recurrence and or spreading of metastases.

### EXAMPLE III

### Prevention of Cancer Development After Biopsy Diagnosis of Cancer In Situ

The tumor suppressor BRCA1 is a major breast and ovarian cancer susceptibility gene. BRCA1 germ line mutations contribute to only 3% of all breast cancers in Caucasians, and somatic mutations are very rare in sporadic breast cancer (Khoo (1999) Oncogene 18:4643) and ovarian cancer (Merajver et al. (1995) Nat. Genet. 9:439).

Several lines of evidence strongly suggest that reduced expression of BRCA1 is involved in the etiology of 30-40% of sporadic breast and ovarian cancer (Taylor et al. (1998) Int. J. Cancer 79:334). Lower or undetectable levels of BRCA1 protein have been observed in sporadic breast cancer, and the majority of high-grade ductal carcinomas express very low levels of the BRCA1 protein as compared to normal mammary tissue and lobular cancers (Wilson et al. (1999) Nat. Genet. 21:236). A similar reduction of the BRCA1 protein has been observed in sporadic ovarian carcinomas. Id.

The BRCA1 gene contains two alternative first exons, 1a and 1b, resulting in two BRCA1 transcripts with different 5′UTR but the same open reading frame for BRCA1 protein. These transcripts, which are formed by the selective use of different promoters, are present at different levels in various normal and tumor tissues and cell lines (Sobczak et al. (2002) J. Biol. Chem. 277:17349; Xu et al. (1995) Hum. Mol. Genet. 4:2259).

Furthermore, comparison of the BRCA1 transcripts expressed in a normal mammary gland with those expressed in breast cancer tissue showed that BRCA1 mRNAb is expressed in breast cancer tissue but not in a normal mammary gland, and that BRCA1 mRNAa is present in both normal and cancer tissue (Xu et al. (1995) Hum. Mol. Genet. 4:2259).

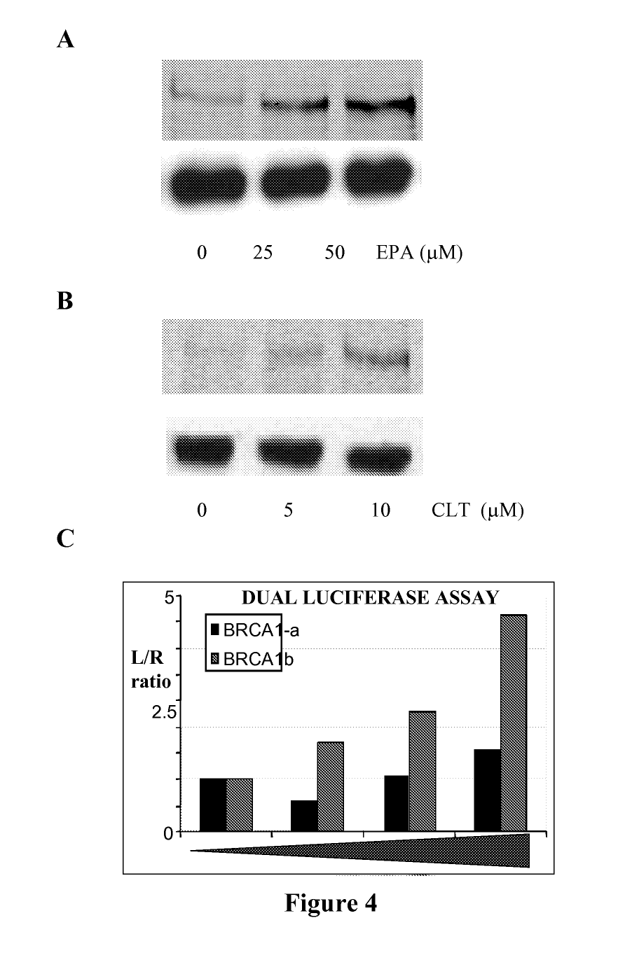

MCF-7 breast cancer cells, known to have very low levels of BRCA1 protein, were exposed to CLT and EPA. FIGS. 4A and 4B show that both drugs induced a significant up-regulation of BRCA1 in a dose-dependent manner. To confirm that up-regulation of BRCA1 was at the level of translation, the 5′ UTRs of either BRCA1 mRNAa or mRNAb were placed in front of the firefly luciferase reporter gene using in both cases renilla luciferase as an internal control. These constructs were transfected into MCF-7 breast cancer cells. CLT and EPA significantly up-regulated translation of the reporter fused to the 5′ UTR of mRNAb. CLT increased the expression of firefly luciferase when its mRNA contained the 5′ UTR of BRCA1 mRNAb (gray), but not the 5′ UTR of BRCA1 mRNAa (black) (FIG. 4C). Without intending to be bound by theory, these experiments indicate that the inhibitors of translation initiation that induce phosphorylation of eIF2α restrict the availability of the ternary complex to translationally up-regulate the tumor suppressor gene BRCA1, a critical player in many types of sporadic breast and ovarian cancers.

In a patient with breast cancer, a biopsy will be performed. If the biopsy shows low levels of BRCA1 and, after treatment for a time interval with an agent described herein (e.g., a translation inhibitor), phosphorylation of eIF2α and/or up-regulation of BRCA1 is detected, prolonged preventive treatment would be given using one or more agents described herein such as EPA, CLT or other translation initiation inhibitors.

### EXAMPLE IV

### Administration of OMEGARX™ Fish Oil Increases the EPA Content of RBC Membranes

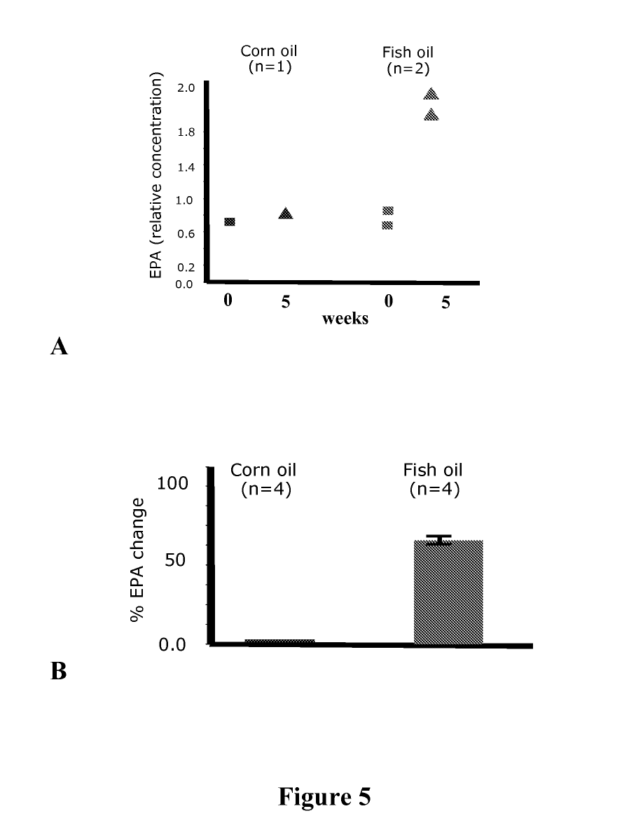

Compliance with oil intake was assessed by comparing the distribution of n-3 and n-6 PUFAs in red blood cell (RBC) membranes before and after the 5 week intervention period (as detailed in Methods). The distribution of fatty acids in RBC membranes is an accepted and validated method to establish the relative composition of dietary fatty acids over an extended period of time (Giovannucci et al. (1993) JNCI Cancer Spectrum 85:1571). The results indicated that daily oral administration of 15 grams of fish oil for 5 weeks increased the levels of the n-3 PUFA EPA in the RBC membranes. No change was observed in the EPA content of RBC membranes in the corn oil subject (FIG. 5A). These results are consistent with those of a preliminary experiment conducted in volunteer subjects who received 10 grams of fish or corn oil for 3 weeks (FIG. 5B). These results indicate that administration of 10-15 grams of fish oil daily for 3-5 weeks is sufficient to elevate the EPA content of RBC membranes.

### EXAMPLE V

### Additional Evidence for Detection of Phosphorylated eIF2α and BiP in Human Subjects

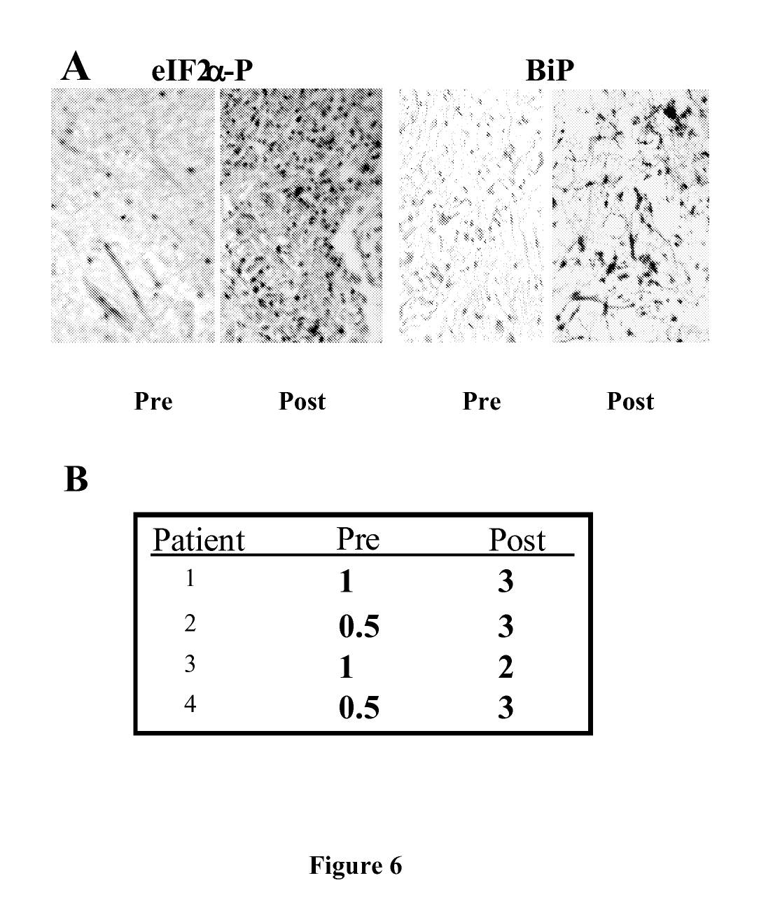

Pathology specimens were obtained from a clinical trial designed to assess the differentiation effect of TRO on human liposarcoma administered between the time of biopsy and the time of surgery (Demetri (1999) Proc. Natl. Acad. Sci. U S A 96:3951). Samples were pre-treatment biopsies and post-treatment surgical specimens from the same patient, fixed and paraffin embedded. Analysis of those samples showed that oral administration of TRO caused significant phosphorylation of eIF2α and BiP expression in the tumors compared with the pre-treatment biopsy samples (FIG. 6A depicts one representative sample, and FIG. 6B depicts the scores of the four patients). The results of these studies in humans show that phosphorylated eIF2α BiP and cyclin D1 are readily detected in human tumors including prostate, and that the treatment regime described herein can induce detectable changes in the fatty acid composition of RBC membranes, the levels of eIF2α phosphorylation, and the expression of translationally regulated proteins.

### EXAMPLE VI

### EPA as an Inhibitor of Translation Initiation

EPA Inhibits Proliferation of Human Prostate Cancer and Other Cancer Cell Lines.

The effect of EPA on the proliferation of a panel of cancer cell lines representative of most common human cancers was studied using the sulfarodamine B cell growth assay (SRB). EPA inhibited the proliferation of all cancer cell lines tested, including androgen dependent and androgen independent prostate cancer cell lines (LNCaP, PC-3 and DU-145) in a dose dependent manner (Table 1: effect of EPA on human cancer cello growth).

TABLE 1

| Cell Line | Origin | IC50 (μM) |
|---|---|---|
| HeLa | Cervix | 10 |
| Hep-G2 | Liver | 24 |
| U-118 MG | Glioblastoma | 36 |
| MMRU | Skin | 36 |
| MDAMB231 | Breast | 37 |
| MCF-7 | Breast | 43 |
| SK-Mel-28 | Skin | 46 |
| HCT-15 | Colon | 47 |
| A549 | Lung | 54 |
| ACHN | Kidney | 62 |
| HT-29 | Colon | 72 |
| Caski-1 | Cervix | 76 |
| HTB-174 | Lung | 79 |
| SK-OV3 | Ovary | 96 |
| DU-145 | Prostate | 36 |
| PC-3 | Prostate | 20 |
| LNCaP | Prostate | 18 |

EPA releases Ca⁺⁺ from intracellular stores

To determine if EPA releases Ca⁺⁺ from intracellular stores of prostate cancer cells, FURA-2-loaded DU-145 cells were challenged with EPA and net changes in the cytoplasmic Ca⁺⁺ concentration [Ca⁺⁺]ᵢ were determined. EPA induced an increase in [Ca⁺⁺]ᵢ in the absence of external Ca⁺⁺. Without intending to be bound by theory, these results indicate that the EPA-induced rise in [Ca⁺⁺]ᵢ was due to release from internal stores because in Ca⁺⁺-free medium these stores are the only source for the increase in cytosolic Ca⁺⁺.

Ca⁺⁺ release from intracellular stores results in the opening of SOC channels in the plasma membrane which allows for the refilling of the stores (Berridge (1995) Biochem. J. 312:1; Gamberucci et al. (1997) Cell Calcium 21:375). Influx through these channels is responsible for the sustained increase in intracellular Ca⁺⁺ observed in response to agonists such as thapsigargin (TG), a potent inhibitor of endoplasmic reticulum and sarcoplasmic reticulum Ca⁺⁺ (SERCA) pumps that release Ca⁺⁺ from intracellular pools. Addition of EPA to TG-treated DU-145 cells rapidly brought the intracellular Ca⁺⁺ towards its basal level in a dose dependent manner. This demonstrates that EPA inhibits SOC channels in prostate cancer cells as it does in other cell lines. Together, these experiments indicate that by releasing calcium from intracellular stores and at the same time inhibiting capacitative Ca⁻⁺ influx through SOC, EPA can partially deplete intracellular calcium stores in prostate cancer cells.

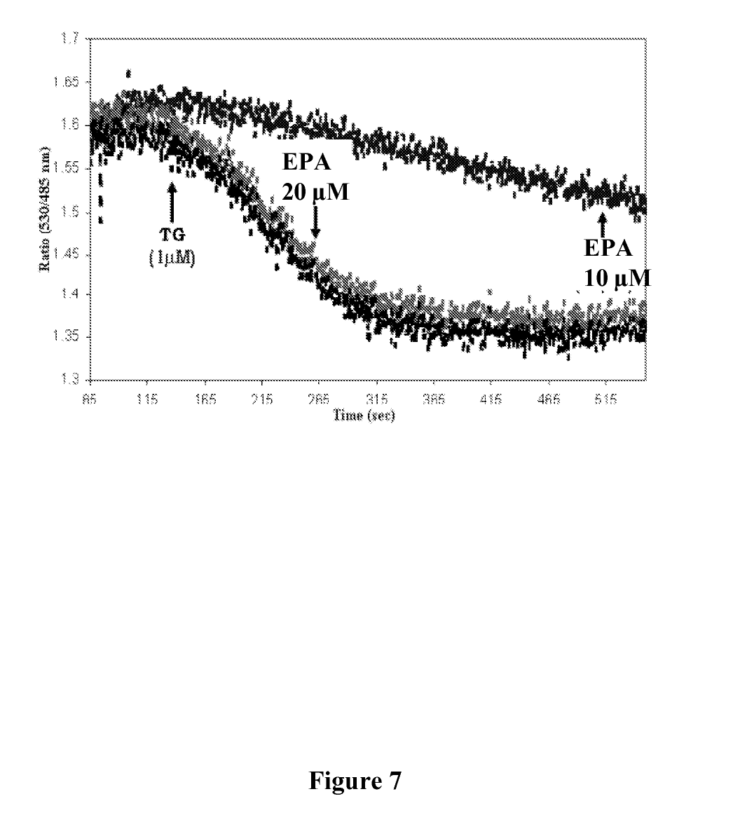

This interpretation was confirmed by transfecting the cancer cells with a recombinant chameleon protein composed of calmodulin attached to cyan fluorescent protein (CFP) on one end and calmodulin-binding peptide attached to yellow fluorescent protein (YFP) on another end, as described by Miyawaki et al. ((1999) Proc. Natl. Acad. Sci. U.S.A. 96:2135). In low or no Ca⁻⁺ medium, this fusion protein is relaxed and the fluorescent proteins are far apart from each other. In contrast, in the presence of calcium, the high affinity of calmodulin for the calmodulin-binding peptide brings both sides of the molecule together and approximates the CFP and YFP in a manner that allows for the transfer of energy between both fluorescent proteins with the corresponding emission of a detectable photon, a phenomenon known as Fluorescence Resonance Energy Transfer (FRET) (Miyawaki et al. (1999) Proc. Natl. Acad. Sci. U.S.A. 96: 2135). A stable cancer cell lines transfected with the chameleon protein construct fused to an ER-localization signal to express the biomarker in the ER (Yu et al. (2000). J. Biol. Chem. 275:23648) was used to demonstrate EPA induced depletion of the ER-calcium stores (FIG. 7).

### EXAMPLE VII

### EPA Activates eIF2α Kinases, Induces Phosphorylation of eIF2α and Inhibits Translation Initiation

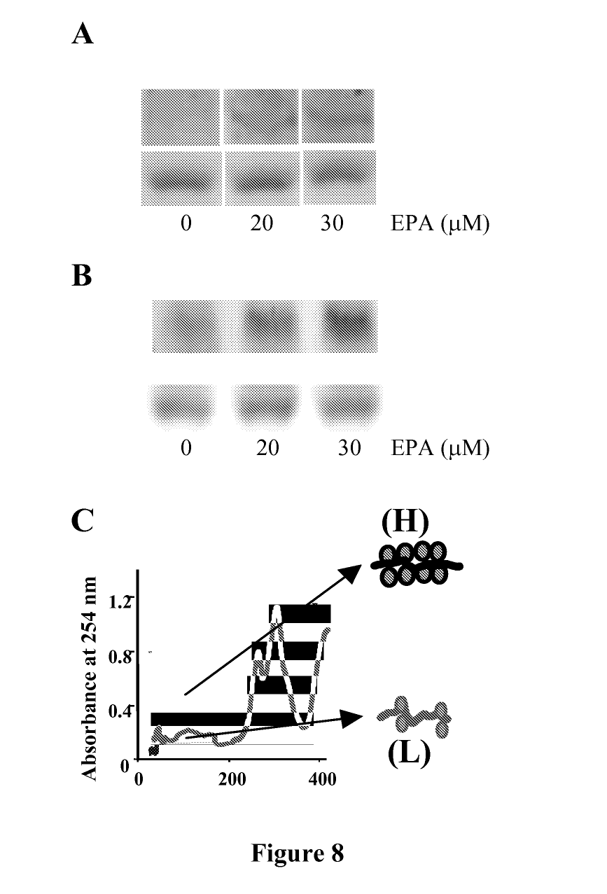

Pharmacological agents that deplete intracellular Ca⁺⁺ stores activate eIF2α kinases (i.e., PKR and/or PERK), resulting in phosphorylation of eIF2α which is responsible for inhibition of translation initiation (Srivastava et al. (1995) J. Biol. Chem. 270:16619). To investigate whether depletion of Ca⁺⁺ stores by EPA causes activation of eIF2α kinases and phosphorylation of eIF2α in prostate cancer cells, DU-145 cells were incubated with or without increasing concentrations of EPA for two hours. The cells were then lysed, and the lysates were immunoblotted with antibodies against the phosphorylated (active) forms of the ER-resident eIF2α-kinase PERK, and also with the anti-S51-P-eIF2α-specific antibody. The results indicated that EPA induces activating phosphorylation of PERK (FIG. 8A), and an approximately 10-fold increase in eIF2α phosphorylation (FIG. 8B). Similar results were obtained in a panel of cancer cells. These results indicate that EPA inhibits translation initiation in prostate cancer cells. This result was further confirmed by determining the polysome profile of EPA-treated human prostate cancer cells by sucrose density gradient centrifugation followed by continuous monitoring at 254 nm during elution. The profiles shown in FIG. 8C demonstrate that EPA significantly shifts the polysome profile from heavy (H) to lighter polysomes (L) and monosomes, the hallmark of inhibition of translation initiation. Similar results were obtained in all cancer cells studied. These results indicate that EPA inhibits translation initiation.

### EXAMPLE VIII

### EPA-Induced GI Cell Cycle Arrest in Prostate Cancer Cells Is Dependent on eIF2α Phosphorylation

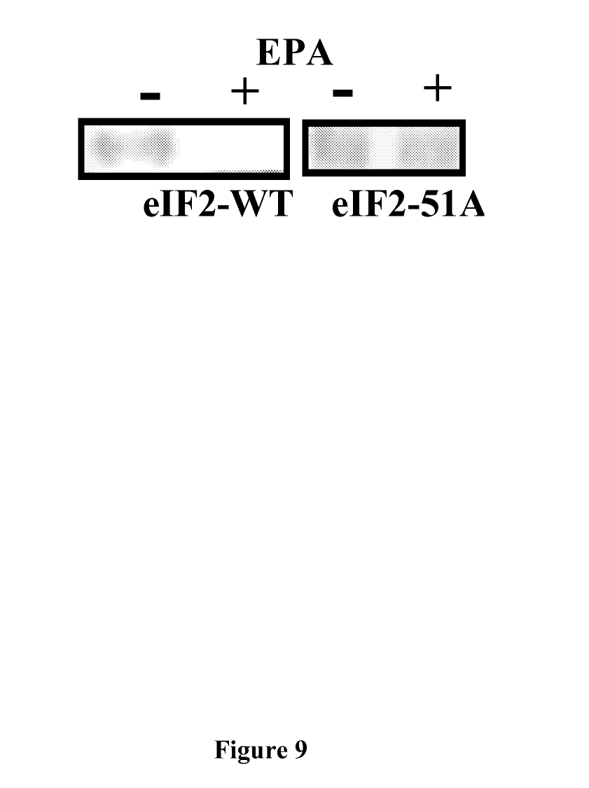

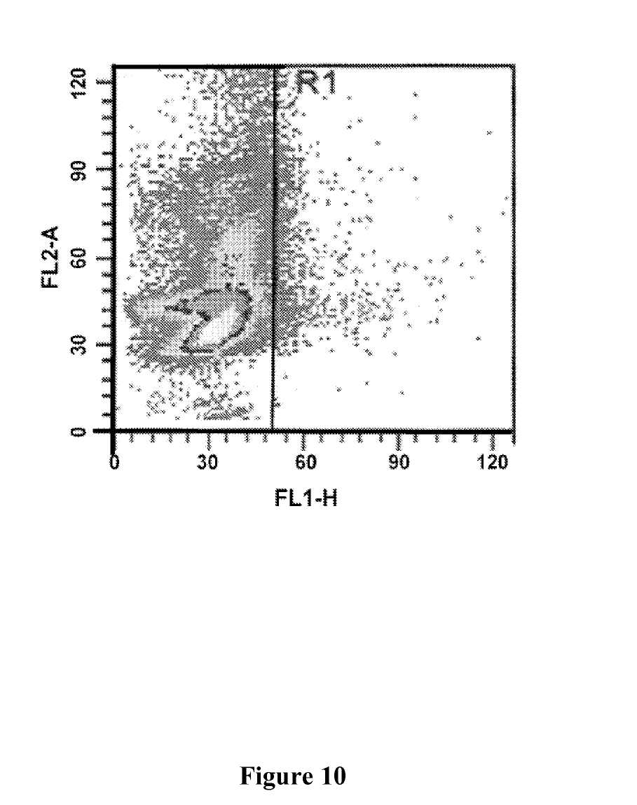

eIF2α51A is a constitutively active but non-phosphorylatable mutant of eIF2α in which serine 51 has been replaced by alanine (FIG. 9). To confirm that eIF2α phosphorylation is also critical for EPA-mediated inhibition of cell proliferation in prostate cancer cells, DU-145 cells were transiently transfected with a construct that co-expresses the fluorescent marker GFP with either eIF2αWT (i.e., eIF2α wild-type) or eIF2α51A. These cells were treated with either EPA or vehicle DMSO, and the cell cycle distribution of GFP expressing cells was determined by FACS analysis. FIG. 10 and Table 2 (cell cycle distribution of GFP-expressing cells in the four groups analyzed) show that EPA induced GI cell cycle arrest in eIF2αWT transfected but not in eIF2α51A transfected DU-145 cells. These experiments demonstrate that in prostate cancer cells, phosphorylation of eIF2α mediates the anti-proliferative effect of EPA, as it does in other cell types.

TABLE 2

| | WT | WT | | 51A | 51A |
|---|---|---|---|---|---|
| WT | Vehicl | EPA | 51A | Vehicl | EPA |
| % G1 | 46 | 67 | % G1 | 43 | 48 |
| % S | 33 | 19 | % S | 34 | 31 |
| % G2/M | 21 | 14 | % G2/M | 23 | 21 |

### EXAMPLE IX

### Inhibition of Translation Initiation Preferentially Abrogates the Expression of G1 Cyclins at the Level of Translation

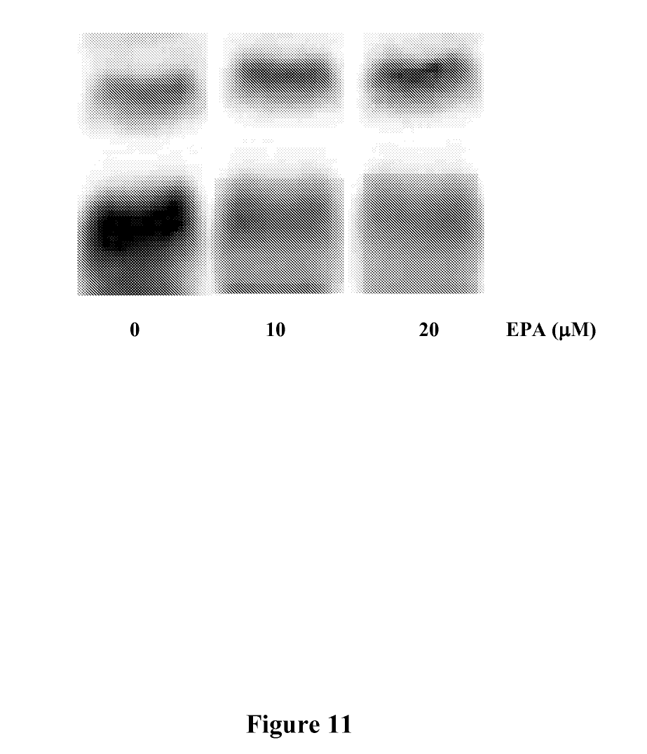

To determine whether EPA down-regulates “weak” mRNAs with complex and stable secondary structure in their 5′ untranslated region (5′ UTR), such as those encoding for oncogenes like G1 cyclins, c-myc, Ras and omitin decarboxylase, the effect of EPA on the levels of cyclin D1 in DU-145 cells was investigated. EPA caused a significant reduction in the expression of cyclin D1 in DU-145 cells in culture (FIG. 11).

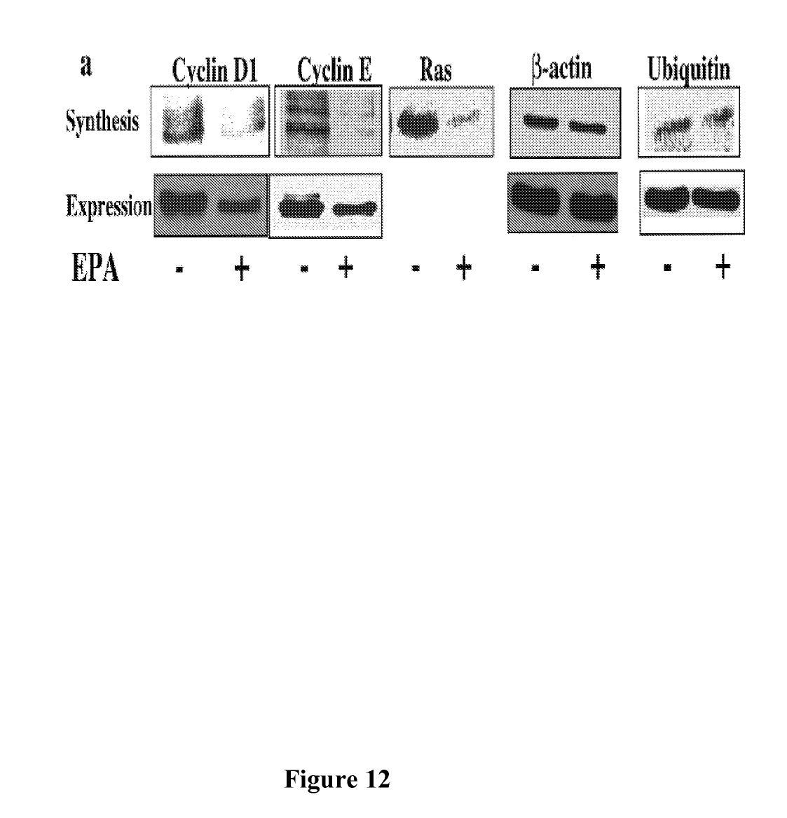

Comparable results were obtained in other cells types, shown in FIG. 12, which demonstrates the preferential effect of EPA on the synthesis and expression of oncogenes (cyclin D1, cyclin E and Ras) with minimal effect on housekeeping proteins such as actin or ubiquitin.

These results indicate that EPA blocks the cell cycle by preventing accumulation of G1 cyclins necessary to drive the progression from G1 into the S phase. These results also demonstrate that inhibition of cyclin D1 expression occurs at the level of translation.

### EXAMPLE X

### EPA induces Up-Regulation of CHOP and BiP in Prostate Cancer Cells

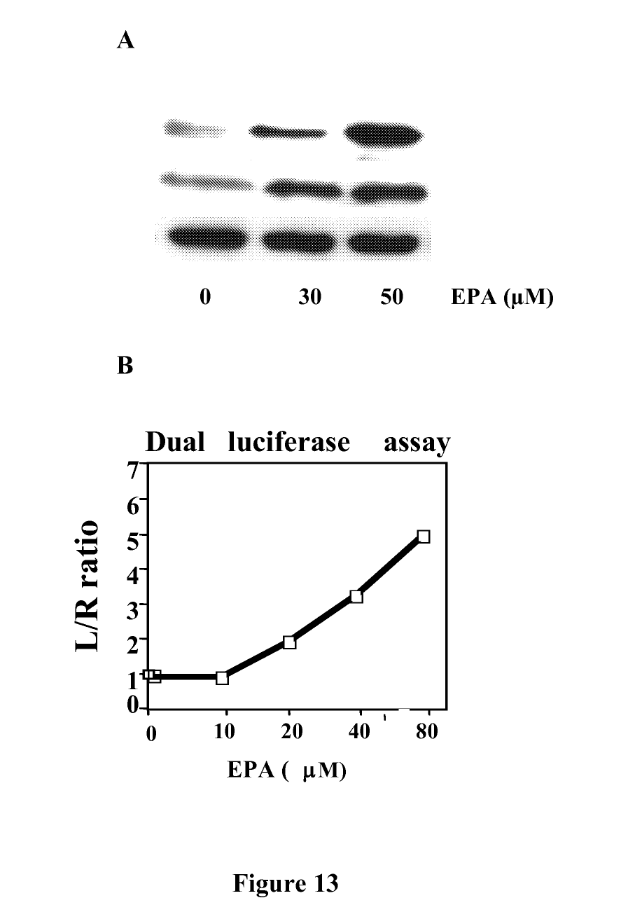

EPA induces the expression of CHOP and BiP in prostate cancer cells (FIG. 13A), as well as in MCF-7 breast cancer and in KLN mouse carcinoma cells. Without intending to be bound by theory, up-regulation of CHOP and BiP are part of the cellular response to EPA in many cancer cell types. CHOP and BiP are members of the gene cluster known as ER-stress response genes, which are under the transcriptional control of the Activating Transcription Factor-4 (ATF-4). Up-regulation of ATF-4 under limited ternary complex conditions is due to peculiar features of its 5′ UTR, which contains several upstream open reading frames (uORFs). These uORFs represent a major barrier to the translational efficiency of ATF-4 when the ternary complex is abundant, but favor its translation when the ternary complex is scarce (Harding et al. (2000) Mol. Cell 6:1099).

Fusion of the 5′ UTR of ATF-4 mRNA to a reporter gene confers onto the reporter mRNAs a similarly high level of translation when the ternary complex is scarce. This property of the ATF-4 5′ UTR was exploited to construct reporter genes that are up-regulated when eIF2α is phosphorylated, and these constructs were used to engineer cancer cells that were used to identify novel chemical entity inhibitors of translation initiation. Treatment of these stably transfected cancer cells with EPA (or other translation initiation inhibitors that phosphorylate eIF2α) induced a several-fold increase in the expression of the reporter gene firefly luciferase (L), whose ORF is preceded by the 5′ UTR of ATF-4, but not of the reporter gene renilla luciferase (R), whose ORF is preceded by a generic 5′ UTR. These cells were used to screen the NCI library of 120,000 compounds, and several promising scaffolds were identified for pre-clinical development as anti-cancer drugs (Fan et al. (2004) BMCL in press; Natarajan et al. (2004) J. Med. Chem. in press; and Natarajan et al. (2004) BMCL in press, herein incorporated herein by reference in their entirety for all purposes). EPA also induced a several-fold increase in the L/R ratios in DU-145 prostate cancer cells transiently transfected with the same reporter gene constructs (FIG. 13B).

### EXAMPLE XI

### Anti-Tumor Effect of EPA in Animal Models of Experimental Cancer

EPA Increases the Life Expectancy of Mice in an Orthotopic Xenograft Model of Human Prostate Cancer

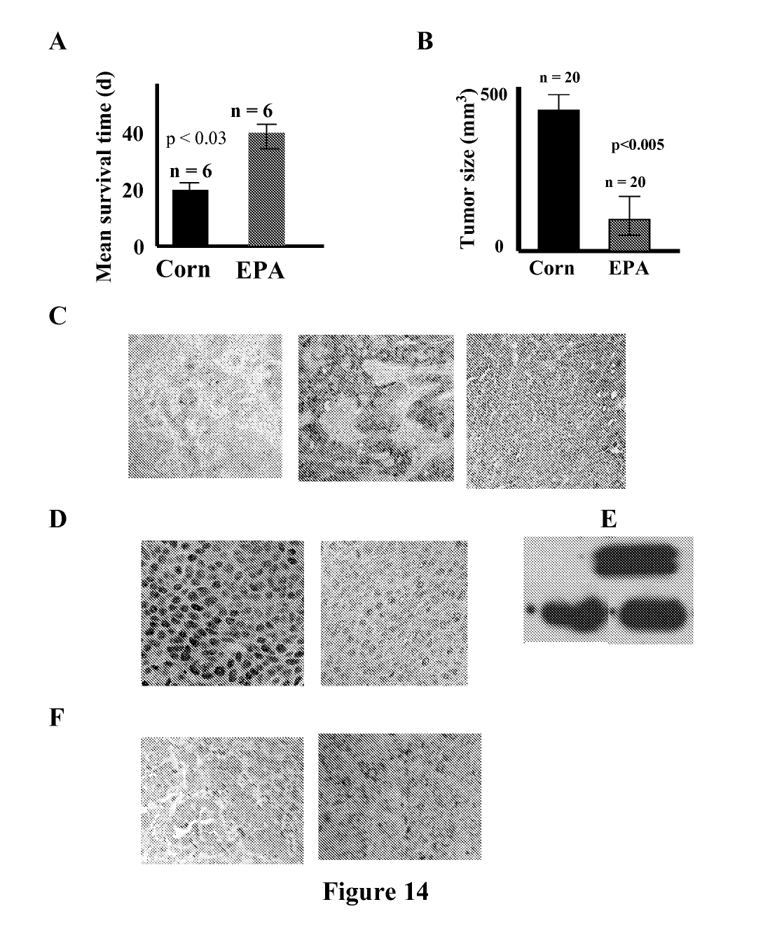

Human PC-3 prostate cancer cells stably transfected with the GFP fluorescent marker were implanted into mouse prostate glands and tumors were allowed to establish. Mice with tumors detectable by whole body fluorescent scanning were treated with EPA or corn oil, orally administered by gavage (2.5 grams/kilogram/day). EPA-treated mice had a 50% longer mean survival time than mice treated with corn oil (38±5 days in EPA vs. 23±3 days in corn oil) (FIG. 14A).

EPA Inhibits Tumor Growth in KLN Mouse Squamous Cell Carcinoma

The anti-cancer action of EPA in vivo was also tested in a syngenic and orthotopic model of mouse KLN-205 mouse squamous cell carcinoma. DBA mice were injected with a total of 2.5×10⁵ KLN-205 cells into the right ventral area and animals were observed for formation of visible tumors before separating them into control and experimental groups. Four days after the implantation of the tumor cells, EPA (2.5 grams/kilogram) or an isocaloric amount of corn oil (control) were administered by gavage and the tumor size was recorded biweekly using calipers. EPA significantly reduced tumor growth of KLN cell squamous cell carcinoma (FIG. 14B). Treatment with EPA induced eIF2α phosphorylation, BiP expression, and down-regulation of cyclin D1 expression (FIGS. 14C-14F). Also, KLN tumors sectioned and re-stained four years after fixation showed no significant reduction in the level of eIF2α phosphorylation (FIG. 14C, EPA2), indicating that this marker is stable over a period of at least four years.

EPA Doubles the Life Expectancy of p53^{-/-} Mice

p53 is the most commonly mutated gene in human cancers. The anti-cancer activity of EPA was investigated in p53^{-/-} mice, which develop a wide variety of spontaneous tumors with 100% penetrance and die from cancer within 50 weeks of age. Age and sex matched animals were randomly distributed to treatment (2.5 grams/kilogram EPA) or control (2.5 grams/kilogram corn oil) administered daily by gavage. Neither control nor EPA treated animals showed any sign of toxicity, which was maintained until the death of the last animal. As shown in FIG. 16, treatment with EPA doubled the life expectancy of p53^{-/-} animals (p<0.005).

### EXAMPLE XII

### Identification of the Direct Molecular Target of EPA and other Ca⁺⁺ Depleting Inhibitors of Translation Initiation

Without intending to be bound by theory, using molecularly engineered cells lacking either one of the three isoforms of the IP3 receptor, the IP3 receptor has been excluded as the molecular target of EPA. Through affinity purification using synthetic compounds amenable to cross-linking and affinity purification in an avidin-biotin system, followed by mass spectrometry sequencing, several potential candidates have been identified, including the ER resident protein calreticulin. Experiments were and will be conducted based on a small interfering RNA (siRNA) gene silencing approach to define further whether one of these candidates is indeed the bona fide target of EPA.

### EXAMPLE XIII

### Inhibition of Translation Initiation Mediates the Anti-Cancer Effect of EPA

EPA Depletes Intracellular Ca²⁻ Stores

Binding of many physiological agonists of cell processes such as hormones, growth factors or cytokines, to their cognate cell membrane receptors induces a transient rise in cytosolic Ca²⁺ following its release from intracellular stores (Halperin, Nutritional Oncology 2d Ed., Herber, Blackburn, Go, Milner, Eds., Section V: Bioactive Food Components and Botanical Approaches to Cancer, Chapter 35: Dietary Lipids and Cancer, in press; incorporated herein by reference in its entirety for all purposes). When Ca²⁺ is released from intracellular stores, Ca²⁺ channels in the plasma membrane, known as store-operated calcium channels (SOC) open to refill the intracellular stores by capacitative Ca²⁺ entry from the extracellular medium thus re-establishing cellular Ca²⁺ homeostasis (Berridge (1995) Biochem J. 312:1; Putney (1997) Cell Calcium 21:257; ).

EPA has a dual effect on intracellular Ca²⁺ homeostasis. On the one hand it induces Ca²⁺ release from the intracellular Ca²⁺ stores, and at the same time inhibits Ca²⁺ influx through SOC in the plasma membrane; these cellular effects require peroxidation of EPA because they are blocked by vitamin E (Palakurthi et al., 2000). By releasing Ca²⁺ from the ER stores while simultaneously closing SOC, EPA partially depletes intracellular Ca²⁺ stores. Depletion of the intracellular Ca²⁺ stores by EPA was confirmed by transfecting cells with ER-targeted “chameleon” proteins that monitor the ER calcium content in real time.

Depletion of intracellular Ca²⁺ stores activates eIF2α kinases and inhibits translation initiation. Inhibition of translation initiation by EPA was demonstrated by sucrose density gradient centrifugation of cell lysates followed by determination of the cell polysome profile. Treatment of cells with EPA shifted the polysome profile from heavy polyribosomal fractions towards light polysomes and free ribosomal subunits (Palakurthi et al. (2000). Cancer Res. 60:2919). This shift of the cell polysome profile towards lighter fractions is recognized as the hallmark of inhibition of translation initiation. Phosphorylation of eIF2α Mediates Inhibition of Translation Initiation by EPA

Inhibition of translation initiation by EPA is mediated by activation of eIF2α kinase-dependent phosphorylation of eIF2α. Without intending to be bound by theory, this conclusion is based on the experimental findings in cancer cell lines treated with EPA: a) EPA causes phosphorylation of eIF2α; b) EPA inhibits translation initiation in wild type cells but not in cells expressing a dominant negative mutant of PKR; and c) cells transfected with a constitutively-active but phosphorylation-resistant mutant of eIF2α (eIF2α-51A) are resistant to the effects of EPA on translation initiation, protein synthesis and cell growth (Palakurthi et al. (2000). Cancer Res. 60:2919).

EPA Downregulates G1 Cyclins and Blocks Cell Cycle Progression in the G1 Phase

Phosphorylation of eIF2α results in preferential downregulation of oncogenes and G1 cyclins. It is well established that several structural features influence the translational efficiency of individual mRNAs. For example, long and complex 5′ UTRs are associated with inefficient translation probably because in the presence of stable secondary structures, ribosomes cannot scan efficiently the entire 5′ UTR to reach the AUG initiation codon (Koromilas et al. (1992) EMBO J. 11:4153-4158; Rousseau et al. (1996). Proc. Natl. Acad. Sci. USA 93:1065). In contrast, mRNAs with simple, less structured 5′ UTRs are translated more efficiently. Interestingly, the leader sequences of approximately 90% of vertebrate mRNAs are between 10 and 200 bases long, mostly without a complex secondary structure and are efficiently translated (“strong” mRNAs).

On the other hand, most mRNAs encoding for cell growth regulatory proteins or proto-oncogenes contain atypical 5′ UTRs which are more than 200 bases long and complex that restrict their translational efficiency and render their translation highly dependent on the activity of translation initiation factors (“weak” mRNAs) (Kozak (199 1). J. Cell Biol. 115:887). Without intending to be bound by theory, this translational inefficiency of proteins that regulate cell proliferation likely plays a crucial role in the maintenance of proper restraints on cell growth; unrestricted translation due to over-expression or disregulation of translation initiation factors mostly increases the expression of oncogenic proteins and results in malignant transformation. For the reasons summarized above, interventions that restrict the rate of translation initiation by targeting translation initiation factors (such as eIF2) preferentially decrease the expression of growth-promoting and oncogenic proteins, and can thereby inhibit the growth and metastatic potential of cancers (Graff et al. (1995) Int. J. Cancer 60:255; Rosenwald et al. (2001) Cancer 92:2164;Willis(1999) Int. J. Biochem. Cell Biol. 31:73).

Consistently, EPA-mediated phosphorylation of eIF2α limits the rate of translation initiation and results in a preferential translational downregulation of G1 cyclins. EPA inhibits the synthesis and expression of cyclin D1, cyclin E and Ras, while minimally affecting the synthesis and expression of housekeeping proteins such as β-actin or ubiquitin. Cyclin D1 expression is down-regulated at the level of translation. When cells were made quiescent by serum withdrawal for 18 hours and then stimulated with basic fibroblast growth factor (bFGF), there was no cyclin D1 mRNA. Eight hours after mitogenic stimulation with bFGF, the expression of cyclin D1 mRNA was fully induced, and the cyclin D1 protein was synthesized at high level. In contrast, cells stimulated with bFGF in the presence of EPA showed full expression of cyclin D1 mRNA but reduced synthesis of cyclin D1 protein. This data confirms that EPA inhibits cyclin D1 synthesis and expression at the level of translation. Importantly, this experiment also shows that EPA does not inhibit the bFGF-induced mitogenic signal upstream from the transcriptional activation of cyclin D1. Furthermore, EPA also down-regulated cyclin D1 expression in the tumors in vivo. Taken together, these data indicate that EPA inhibits preferentially the translation initiation of cell cycle regulatory but not of housekeeping proteins that may account for the potent anti-cancer effects of EPA with low toxicity. Down-regulation of GI cyclins by EPA causes cell cycle arrest in the G1 phase, as would have been expected from an agent that inhibits expression of G1 cyclins (Palakurthi et al. (2000) Cancer Res. 60:2919).

EPA Induces the Expression of ER-Stress Genes including Pro-Apoptotic Proteins

EPA induces the expression of the activating transcription factor-4 (ATF-4) regulated gene cluster. Treatment of cells with EPA limits the availability of the eIF2.GTP.Met-tRNAi ternary complex and decreases the overall rate of translation initiation. Scarcity of the ternary complex has different consequences for the translation of the various mRNAs. Under conditions of limited ternary complex availability, the translation of mRNAs' coding for housekeeping proteins such as β-actin and ubiquitin is minimally affected, while the translation of mRNAs coding for oncogenic proteins such as cyclin D1 is dramatically reduced. Paradoxically, the translational efficiency of another subset of mRNAs is significantly enhanced when the ternary complex is scarce. Among these is the mRNA encoding for activating ATF-4, which regulates the transcription of the ER-stress response gene cluster (Harding et al. (2000) Mol. Cell 6:1099; Scheuner et al. (2001) Mol. Cell 7:1165). The ATF-4 mRNA is more efficiently translated under conditions of limited ternary complex availability because its 5′ UTR contains several upstream open reading frames (uORFs) that render its translation highly inefficient when the ternary complex is abundant but significantly more efficient when the ternary complex is scarce. When the 43S pre-initiation complex binds to the 5′ end of the ATF-4 mRNA, it scans the 5′ UTR and initiates translation at the AUG codon of the first uORF. By recognizing the initiation codon of the first uORF, the ribosomal machinery is primed to also recognize its stop codon and dissociate. However, a small fraction of the 40S ribosomal subunit that remains associated with the mRNA continues scanning towards the 3′ end, restarting initiation and falling off at the subsequent initiation and stop codons, respectively. In summary, the probability of reaching the initiation codon of the ATF-4 bona fide uORF is very low when the ternary complex is abundant. In contrast, when the ternary complex is scarce, the probability that the ribosomal machinery would translate the uORFs is reduced, and the probability that the 43S subunit would reach and translate the bona fide uORF of ATF4 is enhanced several-fold (Harding et al. (2000) Mol. Cell 6:1099). As a consequence, stimuli like EPA treatment that induce phosphorylation of eIF2α limit the availability of the ternary complex, translationally upregulate the expression of ATF-4 and increase the transcription and expression of ATF-4 target genes such as BiP and CHOP. Indeed, treatment of cancer cells with EPA induced the expression of both BiP and pro-apoptotic CHOP, providing a molecular explanation for the increased apoptotic rate reportedly seen in cancer cells upon prolonged treatment with EPA. Together with downregulation of oncogenic proteins and the blockade of cell cycle progression in the GI phase, increased apoptosis would also contribute to the anti-cancer properties of EPA.

### Equivalents

Other embodiments will be evident to those of skill in the art. It should be understood that the foregoing description is provided for clarity only and is merely exemplary. The spirit and scope of the present invention are not limited to the above examples, but are encompassed by the claims. All publications and patent applications cited above are incorporated by reference herein in their entirety for all purposes to the same extent as if each individual publication or patent application were specifically indicated to be so incorporated by reference.

What is claimed:

1. A method for identifying an agent that inhibits aberrant cellular proliferation in a cell comprising the steps of:
i) determining a level of eIF2α phosphorylation in the cell;
ii) contacting the cell with the agent;
iii) determining a level of eIF2α phosphorylation in the cell; and
iv) comparing the level of eIF2α phosphorylation in step i) with the level of eIF2α phosphorylation in step iii),
wherein the level of eIF2a phosphorylation in step iii) is greater than the level of eIF2α phosphorylation in step i) if the agent inhibits aberrant cellular proliferation.

2. The method of claim 1, wherein the agent inhibits translation initiation.

3. The method of claim 1, wherein the agent activates an eIF2α kinase.

4. The method of claim 1, wherein the cell is a cancer cell.

5. The method of claim 1, wherein the cell is from a biological sample.

6. The method of claim 1, wherein the cell is from a mammal.

7. The method of claim 1, wherein eIF2α phosphorylation is detected with a phospho-specific anti-eIF2α antibody.

8. The method of claim 1, wherein the agent is selected from the group consisting of agonist, antagonist, peptidomimetic, protein, peptide, nucleic acid and small molecule.

9. A method for monitoring effectiveness of an agent for treating a disorder associated with aberrant cellular proliferation in a biological sample comprising the steps of:
i) determining a level of eIF2α phosphorylation in the biological sample;
ii) contacting the biological sample with the agent;
iii) determining a level of eIF2α phosphorylation in the biological sample; and
iv) comparing the level of eIF2α phosphorylation in step i) with the level of eIF2α phosphorylation in step iii),
wherein the level of eIF2α phosphorylation in step iii) is greater than the level of eIF2α phosphorylation in step i) if the agent effectively treats the disorder associated with aberrant cellular proliferation.

10. The method of claim 9, wherein the agent inhibits translation initiation.

11. The method of claim 9, wherein the agent activates an eIF2α kinase.

12. The method of claim 9, wherein the biological sample is a tissue culture sample.

13. The method of claim 9, wherein the biological sample is from an organism.

14. The method of claim 9, wherein the biological sample is from an animal model of aberrant cellular proliferation.

15. The method of claim 9, wherein the biological sample is contacted with two or more agents.

16. The method of claim 9, wherein the biological sample is a cell.

17. The method of claim 9, wherein the biological sample is a tissue sample.

18. The method of claim 9, wherein the biological sample is a sample from a subject suffering from a disorder associated with aberrant cellular proliferation.

19. A method for monitoring effectiveness of an agent for treating a disorder associated with aberrant cellular proliferation in a subject in need thereof comprising the steps of:
i) obtaining a first biological sample from the subject;
ii) determining a level of eIF2α phosphorylation in the first biological sample;
iii) administering the agent to the subject;
iv) obtaining a second biological sample from the subject;
iii) determining a level of eIF2α phosphorylation in the second biological sample; and
iv) comparing the level of eIF2α phosphorylation in the first biological sample with the level of eIF2α phosphorylation in the second biological sample, wherein the level of eIF2α phosphorylation in the second biological sample is greater than the level of eIF2α phosphorylation in the first biological sample if the agent effectively treats the disorder associated with aberrant cellular proliferation in the subject in need thereof.

20. The method of claim 19, wherein the first and second biological samples are biological fluid samples, tissue samples or cell samples.

21. The method of claim 19, wherein the subject is a mammal.

22. The method of claim 19, wherein the aberrant cellular proliferation is cancer.

23. The method of claim 22, wherein the cancer is selected from the group consisting of prostate cancer, breast cancer and ovarian cancer.

24. The method of claim 19, wherein the agent inhibits translation initiation.

25. The method of claim 19, wherein the agent activates an eIF2α kinase.

26. The method of claim 19, wherein the agent inhibits tumor growth.

27. The method of claim 19, wherein the agent inhibits tumor metastasis.

28. The method of claim 19, wherein the agent increases the life expectancy of the subject.

29. The method of claim 19, further comprising altering the amount of agent administered to increase or decrease the level of eIF2α phosphorylation in step iii).

30. A method of quantifying aberrant cellular proliferation comprising the steps of:
i) obtaining a test sample;
ii) determining a level of eIF2α phosphorylation in the test sample;
iii) obtaining a standard sample;
iv) determining a level of eIF2α phosphorylation in the standard sample; and
v) comparing the level of eIF2α phosphorylation in the test sample and the level of eIF2α phosphorylation in the standard sample, wherein a degree to which eIF2α phosphorylation in the test sample is greater than the level of eIF2α phosphorylation in the standard corresponds to a degree of aberrant cellular proliferation in the test sample.

31. A kit for determining aberrant cellular proliferation in a sample, comprising:
a compound that detects phosphorylated eIF2α; and
a standard sample.

32. The kit of claim 31, further comprising instructions for use.
